(19) United States
(12) Patent Application Publication
(10) Pub. No.: US 2016/0060460 A1
(43) Pub. Date: Mar. 3, 2016

(54) PROCESS FOR PRODUCING A PARTIALLY BALANCED ACID SOLUTION

(71) Applicant: INVISTA TECHNOLOGIES S.À. R.L., St. Gallen (CH)

(72) Inventor: Robert J. WELCH, Salisbury, MD (US)

(73) Assignee: INVISTA TECHNOLOGIES S.À.R.L., St. Gallen (CH)

(21) Appl. No.: 14/786,651

(22) PCT Filed: Apr. 15, 2014
(86) PCT No.: PCT/US14/34224

§ 371 Date: Oct. 23, 2015

Related U.S. Application Data
(60) Provisional application No. 61/818,033, filed on May 1, 2013.
(60) Provisional application No. 61/917,022, filed on Dec. 17, 2013.

(51) Int. Cl. C08L 77/06; G01N 27/416; G01N 33/44

(52) U.S. Cl. CPC C08L 77/06; G01N 33/442; G01N 27/4167

(57) ABSTRACT

A process for producing a partially balanced acid solution that involves metering dicarboxylic acid powder, based on weight, from a loss-in-weight feeder to a feeding conduit that transfers the dicarboxylic acid powder at a low variability feed rate into an in-line disperser, adding a first feed stream of diamine to the in-line disperser in an amount sufficient to form the partially balanced acid solution having a solids content of less than 60%, and storing the partially balanced acid solution at a temperature to maintain the dissolved dicarboxylic acid and to prevent formation of a slurry. The partially balanced acid solution may be used as a feed solution to prepare a nylon salt solution. Process controls for the process are also disclosed.

### CROSS REFERENCE TO RELATED APPLICATION

This application claims priority to U.S. App. No. 61/818,033, filed May 1, 2013, and claims priority to U.S. App. No. 61/917,022, filed Dec. 17, 2013, the entire contents and disclosures of which are incorporated herein.

### FIELD OF THE INVENTION

The present invention relates to the preparation of liquid partially balanced acid (PBA) solution that is enriched in dicarboxylic acid, and in particular to the preparation of PBA solution that using disperser such as an in-line disperser or a vessel with a disperser head. The process also relates to feed forward process controls and feedback controls for producing a PBA solution and a nylon salt solution from the PBA solution.

### BACKGROUND OF THE INVENTION

Polyamides are commonly used in textiles, apparel, packaging, tire reinforcement, carpets, engineering thermoplastics for molding parts for automobiles, electrical equipment, sports gear, and a wide variety of industrial applications. Nylon is a high performance material used in plastic and fiber applications that demand exceptional durability, heat-resistance and toughness. Aliphatic polyamides, referred to as nylon, may be produced from a salt solution of a dicarboxylic acid and a diamine. The salt solution is evaporated and then heated to cause polymerization. One challenge in this production process is ensuring that there is a consistent molar balance of dicarboxylic acid and diamine in the end polyamide. For example, when producing nylon 6,6 from adipic acid (AA) and hexamethylene diamine (HMD), an inconsistent molar balance adversely decreases the molecular weight and may affect the dyeability of the nylon. The molar balance has been achieved using a batch salt process, but batch processes are not suitable for large industrial production. In addition, the molar balance has been achieved in a continuous mode by multiple reactors, each with a separate charge of diamine during the salt production.

US Pub. No. 2010/0168375 teaches preparing salt solutions of a diamine and of a diacid, more particularly concentrated solutions of hexamethylene diammonium adipate salt, useful starting materials for the production of polyamides, more specifically of PA66. The salt solutions are prepared by mixing a diacid and a diamine, at a salt concentration by weight from 50% to 80%, in a first stage, to provide aqueous solutions of diacid and diamine having a diacid/diamine molar ratio of greater than 1.1 and, in a second stage, adjusting the diacid/diamine molar ratio, by adding diamine, to a value from 0.9 to 1.1, preferably from 0.99 to 1.01, and in fixing the salt concentration by weight by, optionally, adding water thereto. Similarly, US Pub. No. 2012/0046439 teaches preparing a salt solution in multiple stages with two different diacids.

U.S. Pat. No. 4,442,260 teaches a process for making highly concentrated solutions of nylon salt in which the diamine is added in two portions, one before and one after a step in which water is evaporated from a solution of maximum solubility.

U.S. Pat. No. 4,213,884 teaches a process for the manufacture of a highly concentrated aqueous solution of a salt of a dicarboxylic acid and a diamine, as well as of a nylon precondensate, by reacting an alkanedicarboxylic acid of 6 to 12 carbon atoms and a diamine. An aqueous solution, of lower concentration, of a salt of a dicarboxylic acid and a diamine, containing an appropriate dissolved excess of the particular dicarboxylic acid, is reacted with the particular diamine in the molten state, in an equivalent amount to the dissolved dicarboxylic acid, the reaction being carried out under superatmospheric pressure and the final reaction temperature being kept at from 140° C. to 210° C. The solution obtained is used for the manufacture of a nylon.

U.S. Pat. No. 4,131,712 teaches a process for making a high molecular weight polyamide, wherein a diacid-rich component and a diamine-rich component are prepared separately in nonstoichiometric proportions, each of these components melting below the melting temperature of the polyamide product, and preferably below 200° C.; and then the diacid-rich component and the diamine-rich component are contacted in liquid state at high enough temperature to prevent solidification, and in proportions such that the total amounts of diacid and diamine, whether combined or not, are as much as possible stoichiometric.

Other processes have sought to produce anhydrous nylon salt solutions, such as U.S. Pat. Nos. 5,801,278 and 5,674,974; WO99/61510 and EP0411790. It has been observed that complex and time-consuming processes may reduce production rates and have limited application for industrial production of nylon salt solutions. For example, U.S. Pat. No. 6,995,233 describes a continuous process for manufacturing polyamides. The polyamides are of the type obtained from diacids and diamines. The process comprises an operation of continuous mixing of a compound which is rich in amine end groups and a compound which is rich in acid end groups and a polycondensation operation using the mixture. The process relates to the starting phase of such a process, during which an aqueous solution comprising a mixture of monomers in substantially stoichiometric proportions is used. The mixtures constituting the precursors may be anhydrous or may contain up to 10 wt. % water.

Despite efforts to improve the process to achieve target specifications, e.g., proper pH, molar balance, and/or salt concentration in the nylon salt solution, challenges still remain. In particular, dicarboxylic acid, and more specifically adipic acid, is a powder having a variable particle size that leads to wide variations in bulk density and poor flow characteristics. Using a dicarboxylic acid powder introduces another variable that makes it difficult to achieve uniformity of the target specifications in a continuous process. Volumetric feeders for dicarboxylic acid powder amplify this difficulty.

Improvements to control nylon salt uniformity using dicarboxylic acid powder are needed.

### SUMMARY OF THE INVENTION

In a first embodiment, the invention is directed to a process for controlling the continuous preparation of a nylon salt solution comprising generating a model for setting a target feed rate of dicarboxylic acid powder to produce the nylon salt solution having a target pH, controlling feed rate variability of the dicarboxylic acid powder by metering the dicarboxylic acid powder, based on weight, from a loss-in-weight feeder to a feeding conduit that transfers the dicarboxylic acid powder at the target feed rate into a disperser and separately introducing diamine at a first feed rate and water at a second feed rate to the disperser, wherein the first and/or the second feed rates are based on the model, to produce a partially balanced salt solution. In one aspect, the partially balanced acid solution may comprise between 32 wt. % and 46 wt. % dicarboxylic acid, between 11 wt. % and 15 wt. % diamine, and between 39 wt. % and 57 wt. % water. The process further comprises introducing the partially balanced salt solution at a third feed rate, diamine at a fourth feed rate and water at a fifth feed rate to a single continuous stirred tank reactor, wherein the third, fourth and/or fifth feed rates are based on the model, and continuously withdrawing the nylon salt solution from the single continuous stirred tank reactor directly into a storage tank, wherein the withdrawn nylon salt solution has a pH less than ±0.04 of the target pH. The disperser may be an in-line disperser or a vessel with a disperser head. In one embodiment, the target feed rate of dicarboxylic acid powder is based on the production rate for the nylon salt solution. Preferably, the stoichiometric amount of the dicarboxylic acid powder need to produce the nylon salt solution is fed to the disperser. Advantageously no powder needs to be introduced into the single continuous stirred tank reactor. In one embodiment, the process involves maintaining the temperature of the partially balanced acid solution at a temperature between 50° C. and 60° C., preferably between 50° C. and 55° C.

The process may involve feeding at least two streams of diamine, one to the disperser and one to the continuous stirred tank reactor. In one embodiment, the first feed stream of diamine comprises between 15 wt. % and 30 wt. % diamine and between 70 wt. % and 85 wt. % water and the second feed stream of diamine comprises between 20 wt. % and 100 wt. % diamine and between 0 wt. % and 80 wt. % water. More preferably, the first feed stream of diamine comprises between 20 wt. % and 30 wt. % diamine and between 70 wt. % and 80 wt. % water and the second feed stream of diamine comprises between 65 wt. % and 100 wt. % diamine and between 0 wt. % and 35 wt. % water.

In one embodiment, the target pH may be selected from within the range between 7.200 and 7.900. In addition, the target salt concentration may be selected from within the range between 50 wt. % and 65 wt. %.

The process control may also comprise continuously introducing a trim diamine feed at a sixth feed rate to a recirculation loop of the single continuous stirred tank reactor, wherein the sixth feed rate is based on the model. The trim diamine may also further control based on feedback. In other embodiments, there may be a trim water feed to be used to control salt concentration based on feedback. The trim water feed may be fed to the vent condensers or single continuous stirred tank reactor.

In one aspect, using the trim diamine, the process may include detecting a change in pH of the nylon salt solution using an on-line pH measurement of the nylon salt solution downstream of the trim diamine introduction; and adjusting the trim diamine feed rate, i.e. sixth feed rate, in response to the change in pH to produce the nylon salt solution having a pH that varies by less than ±0.04 from the target pH.

In another aspect, using the trim diamine, the process may include obtaining a sample portion of the nylon salt solution downstream of the trim diamine introduction, diluting and cooling the sample portion to form a diluted nylon salt solution having a concentration between 5% and 15% and a temperature between 15° C. and 40° C., detecting a change in pH of the diluted nylon salt solution using an on-line pH measurement of the nylon salt solution downstream of the trim diamine introduction, and adjusting the sixth feed rate in response to the change in pH from the diluted nylon salt solution.

In yet another aspect, using the trim diamine, the process may include removing a sample from the nylon salt solution downstream of the trim diamine introduction for an off-line pH measurement of the nylon salt solution in an aqueous solution at a temperature between 15 and 40° C., determining a bias of an on-line pH measurement with the off-line pH measurement, detecting a change in pH of the nylon salt solution using the biased on-line pH measurement of the nylon salt solution downstream of the trim diamine introduction, and adjusting the sixth feed rate in response to the change in pH to produce the nylon salt solution having a pH that varies by less than ±0.04 from the target pH.

It should be understood that these process controls based on feedback may be used in combination and may also be used with a trim water feed to control salt concentrations. In one exemplary embodiment, the process may further comprise producing the nylon salt solution having a target salt concentration that is selected from within the range between 50 wt. % and 65 wt. % comprising the steps of measuring the salt concentration in the nylon salt solution in the recirculation loop with one or more refractometers downstream of the trim diamine introduction, and adjusting the water feed rate, i.e. fifth feed rate, to control salt concentration of the nylon salt solution based on a target salt concentration, wherein the salt concentration of the nylon salt solution varies by less than ±0.5% from the target salt concentration.

In a second embodiment, the invention is directed to a process for producing a partially balanced acid solution comprising a) controlling feed rate variability of the dicarboxylic acid powder by metering the dicarboxylic acid powder, based on weight, from a loss-in-weight feeder to a feeding conduit that transfers the dicarboxylic acid powder into a disperser, b) adding a first feed stream of diamine to form a partially balanced acid solution comprising between 32 wt. % and 46 wt % dicarboxylic acid, between 11 wt. % and 15 wt. % diamine, and between 39 wt. % and 57 wt. % water, and c) storing the partially balanced acid solution at a temperature between 50° C. and 60° C. to maintain the dissolved dicarboxylic acid and to prevent formation of a slurry. The disperser may be an in-line disperser or a vessel with a disperser head.

In a third embodiment, the invention is directed to a process device for producing a nylon salt solution comprising a loss-in-weight feeder comprising a hopper, a feeding conduit, and a duct for connecting the hopper and the feeding conduit, wherein the hopper comprises at least one external weight measurement subsystem for controlling a replenishment phase and a feed phase, and at least one lower opening for dispensing a dicarboxylic acid powder during the feed phase, wherein the at least one lower opening is positioned above the feeding conduit and wherein the feeding conduit receives the dicarboxylic acid powder and transfers the dicarboxylic acid powder via at least one rotating auger through an outlet. The process device further comprises a vessel comprising one or more disperser heads, a first recirculation loop, a first inlet that is connected to the outlet of the feeding conduit and a second inlet for introducing a first feed stream of diamine to form a dispersion, wherein the first recirculation loop comprises an in-line mixer and level control valve, a storage tank for storing the dispersion at temperature between 50° C. and 60° C., wherein the storage tank comprises a second recirculation loop connected to the level control valve to receive the dispersion from the vessel, and a continuous stirred tank reactor for receiving a portion of the stored dispersion and a second feed stream of diamine to produce the nylon salt solution.

In a fourth embodiment, the invention is directed to a process device for producing a nylon salt solution comprising a loss-in-weight feeder comprising a hopper, a feeding conduit, and a duct for connecting the hopper and the feeding conduit, wherein the hopper comprises at least one external weight measurement subsystem for controlling a replenishment phase and a feed phase, and at least one lower opening for dispensing a dicarboxylic acid powder during the feed phase, wherein the at least one lower opening is positioned above the feeding conduit and wherein the feeding conduit receives the dicarboxylic acid powder and transfers the dicarboxylic acid powder via at least one rotating auger through an outlet. The process device further comprises an in-line disperser having a first inlet that is connected to the outlet of the feeding conduit, a second inlet for introducing a first feed stream of diamine to form a dispersion, and a disperser outlet; a storage tank for storing the dispersion at temperature between 50° C. and 60° C., wherein the storage tank comprises a recirculation loop connected to the disperser outlet to receive the dispersion; and a continuous stirred tank reactor for receiving a portion of the stored dispersion and a second feed stream of diamine to produce the nylon salt solution.

In a sixth embodiment, the invention is directed to a process for polymerization of the nylon salt solution comprising adipic acid and hexamethylene diamine to form nylon 6,6 comprising evaporating the nylon salt solution to form a concentrated stream, and polymerizing the concentrated stream in a second reactor to form a polyamide product. The nylon salt solution is prepared from a PBA solution as described herein. In one embodiment, a portion of the partially balanced acid solution may be introduced into the polymerization reactor.

### BRIEF DESCRIPTION OF THE DRAWINGS

The present invention will be better understood in view of the appended non-limiting figures, in which:

FIG. 1 is a general flow process for producing a nylon salt solution in accordance with an embodiment of the present invention.

FIG. 2A is a schematic diagram of a loss-in-weight feeder and in-line disperser for producing a partially balanced acid solution enriched in AA in accordance with an embodiment of the present invention.

FIG. 2B is a schematic diagram of loss-in-weight feeder and vessel having a disperser head for producing a PBA solution enriched in AA in accordance with an embodiment of the present invention.

FIG. 3 is a schematic diagram of a continuous stirred tank reactor in accordance with an embodiment of the present invention.

FIG. 4 is a schematic diagram having process controls in accordance with an embodiment of the present invention.

FIG. 5 is a schematic diagram for a nylon 6,6 production process in accordance with an embodiment of the present invention.

FIGS. 6-8 are graphs showing the feed rate variability of adipic acid from a loss-in-weight feeder in accordance with an embodiment of the present invention.

### DETAILED DESCRIPTION OF THE INVENTION

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, group of elements, components, and/or groups thereof.

Language such as “including,” “comprising,” “having,” “containing,” or “involving,” and variations thereof, is intended to be broad and encompass the subject matter listed thereafter, as well as equivalents, and additional subject matter not recited. Further, whenever a composition, a group of elements, process or method steps, or any other expression is preceded by the transitional phrase “comprising,” “including,” or “containing,” it is understood that it is also contemplated herein the same composition, group of elements, process or method steps or any other expression with transitional phrases “consisting essentially of,” “consisting of,” or “selected from the group of consisting of,” preceding the recitation of the composition, the group of elements, process or method steps or any other expression.

The corresponding structures, materials, acts, and equivalents of all means or step plus function elements in the claims, if applicable, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment(s) was/were chosen and described in order to best explain the principles of the invention and the practical application, and to enable others of ordinary skill in the art to understand the invention for various embodiments with various modifications as are suited to the particular use contemplated. Accordingly, while the invention has been described in terms of embodiments, those of skill in the art will recognize that the invention can be practiced with modifications and in the spirit and scope of the appended claims.

Reference will now be made in detail to certain disclosed subject matter. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which can be included within the scope of the presently disclosed subject matter as defined by the claims.

### Introduction

The present invention is generally directed to production of nylon salt solution and polyamides produced from the nylon salt solution of a dicarboxylic acid and a diamine. In particular, the present invention is related to producing a liquid partially balanced acid (PBA) solution enriched in dicarboxylic acid, also referred to as an acid rich feed, that is used as a feed solution to form a nylon salt solution. The nylon salt solution is formed to achieve a target salt concentration and/or target pH. The PBA solution is partially balanced and does not achieve the target pH or the target salt concentration of the nylon salt solution. The PBA solution may be combined with another feed of diamine and water in a single continuous stirred tank reactor to achieve the targets to produce the nylon salt solution having uniform pH. The PBA solution advantageously allows introduction of the dicarboxylic acid in a liquid phase into the single continuous stirred tank reactor. In one embodiment, the nylon salt solution having uniform pH may be polymerized to form nylon 6,6. Other types of polyamides may be produced depending on the starting monomers used.

In the description below, the terms adipic acid (AA) and hexamethylene diamine (HMD) will be used to denote the dicarboxylic acid and the diamine. When adipic acid is used the PBA solution is a partially balanced adipic solution. However, this process also applies to other dicarboxylic acids and other diamines indicated herein.

Dicarboxylic acids suitable for the present invention are selected from the group consisting of oxalic acid, malonic acid, succinic acid, glutaric acid, pimelic acid, adipic acid, suberic acid, azelaic acid, sebacic acid, undecanedioic acid, dodecandioic acid, maleic acid, glutaconic acid, traumatic acid, and muconic acid, 1,2- or 1,3-cyclohexane dicarboxylic acids, 1,2- or 1,3-phenylenediacetic acids, 1,2- or 1,3-cyclohexane diacetic acids, isophthalic acid, terephthalic acid, 4,4′-oxybisbenzoic acid, 4,4-benzophenone dicarboxylic acid, 2,6-napthalene dicarboxylic acid, p-t-butyl isophthalic acid and 2,5-furandicarboxylic acid, and mixtures thereof. In one embodiment, the dicarboxylic acid monomer comprises at least 80% adipic acid, e.g., at least 95% adipic acid.

For making nylon 6,6, adipic acid (AA) is the most suitable dicarboxylic acid and is used in the powder form. AA generally is available in a pure form containing very low amounts of impurities. Typical impurities include other acids (monobasic acids and lower dibasic acids), less than 60 ppm, nitrogenous materials, trace metals such as iron (less than 2 ppm) and other heavy metals (less than 10 ppm or less than 5 ppm), arsenic (less than 3 ppm) and hydrocarbon oil (less than 10 ppm or less than 5 ppm).

Diamines suitable for the present invention are selected from the group consisting of ethanol diamine, trimethylene diamine, putrescine, cadaverine, hexamethylene diamine, 2-methyl pentamethylene diamine, heptamethylene diamine, 2-methyl hexamethylene diamine, 3-methyl hexamethylene diamine, 2,2-dimethyl pentamethylene diamine, octamethylene diamine, 2,5-dimethyl hexamethylene diamine, nonamethylene diamine, 2,2,4- and 2,4,4-trimethyl hexamethylene diamines, decamethylene diamine, 5-methylnonane diamine, isophorone diamine, undecamethylene diamine, dodecamethylene diamine, 2,2,7,7-tetramethyl octamethylene diamine, bis(p-aminocyclohexyl)methane, bis(aminomethyl)norbornane, C₂-C₁₆ aliphatic diamine optionally substituted with one or more C₁ to C₄ alkyl groups, aliphatic polyether diamines and furanic diamines, such as 2,5-bis(aminomethyl)furan, and mixtures thereof. The diamine selected may have a boiling point higher than the dicarboxylic acid, and the diamine is preferably not xylylenediamine. In one embodiment, the diamine monomer comprises at least 80% hexamethylene diamine, e.g., at least 95% hexamethylene diamine. Hexamethylene diamine (HMD) is most commonly used to prepare nylon 6,6. HMD solidifies at about 40° C. to about 42° C. and water is commonly added to depress this melt temperature and ease handling. Thus, HMD may be commercially available as a concentrated solution, e.g., from 80 wt. % to 100 wt. % or from 92 wt. % to 98 wt. %.

In addition to polyamides based solely on dicarboxylic acids and diamines, it is sometimes advantageous to incorporate other monomers. When added at proportions of less than 20 wt. %, e.g., less than 15 wt. %, these monomers may be added into the nylon salt solution without departing from this invention. Such monomers may include monofunctional carboxylic acids such as formic acid, acetic acid, propionic acid, butyric acid, valeric acid, benzoic acid, caproic acid, enanthic acid, octanoic acid, pelargonic acid, capric acid, undecanoic acid, lauric acid, myristic acid, myristoleic acid, palmitic acid, palmitoleic acid, sapienic acid, stearic acid, oleic acid, elaidic acid, vaccenic acid, linoleic acid, erucic acid and the like. These may also include lactams such as α-acetolactam, α-propiolactam, β-propiolactam, γ-butyrolactam, δ-valerolactam, γ-valerolactam, caprolactam and the like. These may also include lactones such as α-acetolactone, α-propiolactone, β-propiolactone, γ-butyrolactone, δ-valerolactone, γ-valerolactone, caprolactone, and such like. These may include difunctional alcohols such as monoethylene glycol, diethylene glycol, 1,2-propanediol, 1,3-propanediol, dipropylene glycol, 1,2-butanediol, 1,3-butanediol, 1,4-butanediol, 2,3-butanediol, 1,2-pentanediol, 1,5-pentanediol, etohexadiol, p-menthane-3,8-diol, 2-methyle-2,4-pentanediol, 1,6-hexanediol, 1,7-heptanediol, and 1,8-octanediol. Higher functionality molecules such as glycerin, trimethylolpropane, triethanolamine and the like may also be useful. Suitable hydroxylamines may also be selected such as ethanolamine, diethanolamine, 3-amino-1-propanol, 1-amino-2-propanol, 4-amino-1-butanol, 3-amino-1-butanol, 2-amino-1-butanol, 4-amino-2-butanol, pentanolamine, hexanaolamine, and the like. It will be understood that blends of any of these monomers may also be used without departing from this invention.

It is also sometimes advantageous to incorporate other additives into the polymerization process. These additives may include heat stabilizers such as copper salts, potassium iodide, or any of the other antioxidants known in the art. Such additives may also include polymerization catalysts such as metal oxides, acidic compounds, metal salts of oxygenated phosphorous compounds or others known in the art. Such additives may also be delustrants and colorants such as titanium dioxide, carbon black, or other pigments, dyes and colorants known in the art. Additives used may also include antifoam agents such as silica dispersions, silicone copolymers, or other antifoams known in the art. Lubricant aids such as zinc stearate, stearylerucamide, stearyl alcohol, aluminum distearate, ethylenebisstearamide or other polymer lubricants known in the art may be used. Nucleating agents may be included in the mixtures such as fumed silica or alumina, molybdenum disulfide, talc, graphite, calcium fluoride, salts of phenylphosphinate or other aids known in the art. Other common additives known in the art such as flame retardants, plasticizers, impact modifiers, and some types of fillers may also be added into the polymerization process.

The present invention advantageously achieves a nylon salt solution comprising an AA/HMD salt having a target pH. In particular, the present invention achieves the target pH using a fewer number of vessels than conventional processes, and in particular, achieves the target pH in a single reactor, e.g., a single continuous stirred tank reactor (CSTR) where formation of the nylon salt solution occurs. In the present application, the nylon salt solution is prepared using a disperser and a single continuous stirred tank reactor that can achieve higher production rates than batch processes. In a batch process, the amount of time and capital costs for equipment to achieve production rates similar to those achievable with a continuous process make a batch process impractical. The target pH may be any pH value chosen by one skilled in the art and may be selected based on the desired end polymer product. Without being bound by theory, the target may be selected from the highest inflection slope of the pH curve, at a level that is optimum for the range of intended polymer products.

In some exemplary embodiments, the target pH of the nylon salt solution may be a value within the range between 7.200 and 7.900, e.g., preferably between 7.400 and 7.700. The variation of the actual pH of the nylon salt solution from the target pH of the nylon salt solution may be less than ±0.04, more preferably less than ±0.03, and most preferably less than ±0.015. Thus, for example, if the target pH is 7.500, then the pH of the nylon salt solution is between 7.460 and 7.540, and more preferably between 7.470 and 7.530. For purposes of the present invention, variability of pH refers to the average variation over a continuous operation. This variation is very low, less than ±0.53%, and more preferably less than ±0.4%, and produces a nylon salt solution with a uniform pH. A uniform nylon salt solution that has a low variability from the target pH is beneficial to improve the reliability of the polymerization process to produce a uniform, high quality polymer product. The nylon salt solution having a uniform pH also allows for a consistent quality feed to the polymerization process. The target pH may vary depending on the manufacturing site. Generally, a pH of 7.620, as measured at 9.5% salt concentration at 25° C., produces a nylon salt solution having a molar ratio of AA to HMD of 1, based on the free and chemically combined AA and HMD. For purposes of the present invention, the molar ratio may vary within the range of 0.8:1.2 depending on the target pH. Having a uniform pH also means that the molar ratio of the nylon salt solution has a corresponding low variability.

In addition to the target pH, the present invention may also achieve a target salt concentration. The target salt concentration may be any salt concentration chosen by one skilled in the art and may be selected based on the desired end polymer product and storage considerations. The water concentration of the nylon salt solution may be between 35 wt. % and 50 wt. %. The nylon salt solution may have a salt concentration between 50 and 65 wt. % e.g., between 60 and 65 wt. %. The variability of the salt concentration of the nylon salt solution is preferably very low, e.g., less than ±0.5% from a target salt concentration, less than ±0.3%, less than ±0.2%, or less than ±0.1%. For purposes of the present invention, variability of salt concentration refers to the average variation over a continuous operation. Thus, for example, if the target salt concentration is 60%, then a uniform nylon salt solution has a salt concentration between 59.5 wt. % and 60.5 wt. %, preferably between 59.7 wt. % and 60.3 wt. % and more preferably between 59.9 wt. % and 60.1 wt. %. The target salt concentration may vary depending on the manufacturing site.

The nylon salt solution may be stored as a liquid at a temperature of less than 110° C. at atmospheric pressure, e.g., between 60° C. and 110° C., or between 100° C. and 105° C. Concentrations above 65 wt. % require higher temperature and may require pressurization to maintain the nylon salt solution as a liquid, e.g., a homogeneous liquid. The salt concentration may affect the storage temperature and generally it is efficient to store the nylon salt solution at a lower temperature and at atmospheric pressure. However, lower salt concentrations undesirably increase the energy consumption to concentrate the nylon salt solution prior to polymerization.

The present invention uses a PBA solution for introducing AA into the nylon salt solution and the PBA solution does not achieve the target pH or target salt concentration of the nylon salt solution. Preferably the entire amount of AA required for the nylon salt solution is introduced into the PBA solution that achieves a low variability of AA concentration of less than ±5%, e.g, preferably less than ±2%, less than ±1%, or less than ±0.5%.

The temperature of the nylon salt solution is controlled independently from the molar ratio of the AA to HMD. Although the molar ratio and concentration of solids in the nylon salt solution affect temperature of the nylon salt solution, the process relies on heat exchangers, coils and/or a jacketed CSTR to remove heat from the process and thus control the temperature of the nylon salt solution. The temperature of the nylon salt solution may be controlled to vary by less than ±1° C. from a desired temperature. The temperature of the nylon salt solution is selected to be below the boiling point of the nylon salt solution but above the crystallization temperature. For example, a nylon salt solution with a solids concentration of 63% has a boiling point from 108° C. to 110° C. at atmospheric pressure. Therefore, the temperature is controlled to be less than 110° C., e.g., less than 108° C., but above the crystallization temperature.

Prior art solutions to achieving low variability in the nylon salt focus on adjusting the AA:HMD molar ratio and HMD concentration in the salt solution using multiple reactors. This focus is due, at least in part, to variability in the AA powder bulk density and poor flowability characteristics, leading to an inherent unpredictability of an AA powder feed. The variability in the AA powder bulk density is amplified when using a volumetric feeder for feeding the AA powder to the reactor(s). Due to AA's high melt temperature, AA is typically supplied as a powder, which increases the difficulty of handling AA. To decrease the difficulty of handling AA powder, the present invention forms a liquid PBA solution that comprises AA. The PBA solution is prepared by combining AA powder with liquid diamine AA powder typically has an average size that varies between 75 and 500 microns, e.g., between 100 and 300 microns. The finer powder has substantially more surface area and particle contact which leads to clumping. Preferably, AA powder contains less than 20% of particulates that are less than 75 microns, e.g., less than 10%. Because the AA powder is generally metered on a volumetric basis directly to the reactor in powder form, variations in powder size affect bulk packing and density of the AA powder fed to the nylon salt reactor. These variations in bulk packing and density then lead to variations in pH and in the molar ratio of AA to HMD in the nylon salt solution. To account for this variation, the prior art solution was to arrange nylon salt reactors in series. See, for example, U.S. Publication Nos. 2012/0046439 and 2010/0168375. This conventional approach uses measurements of the target specifications and feeds monomers within the series of reactors. However, this process requires numerous reactors, measurement, and adjustments that increase cost and limit production rates. Additionally, this conventional approach may be more suited to a batch process than to a continuous process. Finally, these conventional approaches cannot use a model to predict pH and/or salt concentration, and thus adjustments are constantly made to bring the nylon salt solution into target specifications.

The role of particle size and particle size distribution associated with feeding AA powder to a nylon salt process was addressed in the prior art by using multiple reactors to add AA and HMD. It has been found that by metering the AA powder on a weight basis instead of a volumetric basis, the variability of the AA powder feed rate may be greatly reduced. In some aspects, the AA powder feed rate may vary by less than ±5% from the target AA powder feed rate, e.g., less than ±3% or less than ±1%. With this stable feed, the disclosed process allows for the use of one single reactor, instead of numerous reactors in series, to form a nylon salt solution to target specifications. It is difficult to control the variation of the nylon salt solution from target pH and target salt concentration using a single reactor operating under high continuous production rates without a stable feed of AA powder because there is a limit on the ability to adjust the monomers. Having a stable feed of AA powder allows the process controls to take advantage of feed forward rates for HMD and allows for adjusting trim HMD to adjust the pH to achieve the target pH. Advantageously, the contemplated embodiments provide simpler designs than previous disclosures by reducing the number of unit operations in the process. Thus, the disclosed process omits steps previously believed necessary. This reduces plant footprint and capital costs. The resulting nylon salt solution may then be polymerized to form the desired polyamide.

To achieve acceptable production in the industrial manufacture of nylon salts, a continuous process may be used to produce the nylon salt solution that achieves the target pH and target salt concentration. A batch process would require significantly larger vessels and reactors that would not be comparable with the production rates achievable by smaller continuous production equipment. It is beneficial in the polymerization to start with a nylon salt solution having uniformity in both the pH and salt concentration. Slight variations can cause production quality problems with the polymerization that require additional monitoring, control, and adjustment of the polymer process.

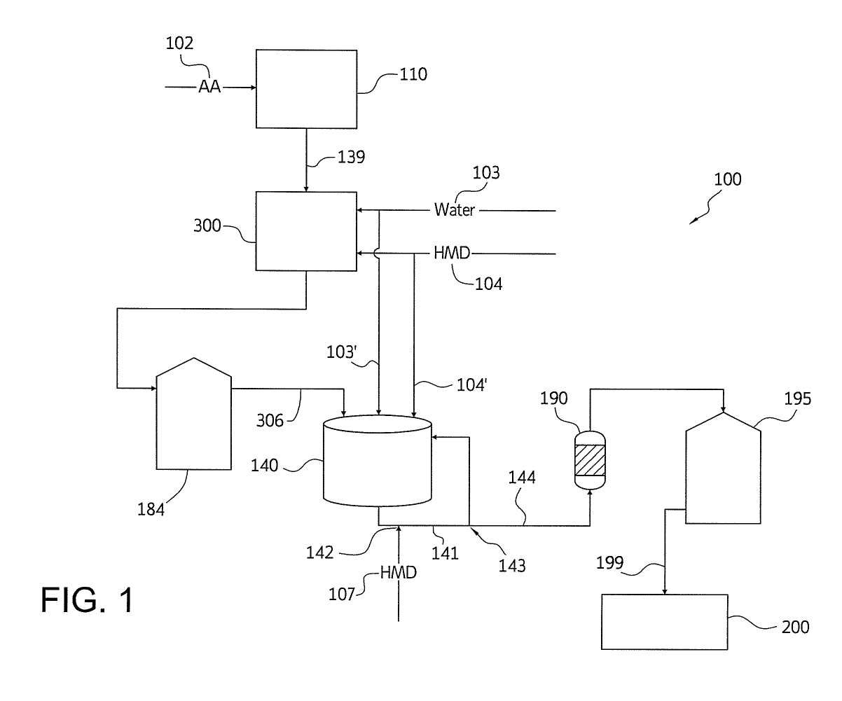

FIG. 1 provides a general outline of the process to produce the nylon salt solution according to embodiments of the present invention. As shown in FIG. 1, a nylon salt solution production process 100 comprises feeding AA powder 102 to a loss-in-weight feeder 110, which produces a metered AA feed 139 that is directed to disperser 300. As discussed further herein, disperser may be an in-line disperser or a vessel with a disperser head. Water via line 103 and HMD via line 104 are also directed to disperser 300 to form a partially balanced acid (PBA) solution 306 that is enriched in AA, which may also be referred as a feed solution. In one embodiment, PBA solution 306 has a molar ratio of AA to HMD between 2:1 and 5:1, e.g., between 2:1 and 3:1, based on free and chemically bonded AA and HMD. PBA solution 306 is kept in a liquid phase and does not form a slurry or solid. As discussed further below, PBA solution 306 stored in tank 184 prior to being introduced to the continuous stirred tank reactor 140. This may allow more mixing, allows for an inventory of AA to be stored prior to forming the nylon salt solution, and allows for independent production of the PBA solution and nylon salt solution. By using liquid PBA solution 306, AA powder 102 is not introduced directly to continuous stirred tank reactor 140. PBA solution 306 is transferred to continuous stirred tank reactor 140. Additionally, water via line 103′ and HMD via line 104′ are fed to continuous stirred tank reactor 140. In some embodiments, lines 103′ and 104′ may be combined (not shown) prior to being fed to reactor 140.

A liquid comprising the nylon salt solution is withdrawn from reactor 140 via recirculation loop 141 and returned to reactor 140. As needed, additional HMD, referred to herein as trim HMD, may be added to the liquid from line 107 at junction 142 to adjust the pH of the nylon salt. The nylon salt solution is withdrawn at junction 143 from the recirculation loop 141 into the conduit 144. The nylon salt solution in conduit 144 passes through filters 190 to remove impurities and is collected in storage tank 195. Similar to PBA solution 306, the nylon salt solution in storage tank 195 does not form a slurry or solid. Generally, these impurities may include corrosion metals and may include impurities from the monomer feeds such as AA powder 102. The nylon salt solution is removed via line 199 to polymerization process 200. The nylon salt solution may be kept in storage tank 195 until needed for polymerization. In some embodiments, storage tank 195 may be transportable.

### Nylon Salt Solution Equipment

In one embodiment, the present invention is directed to a continuous process for producing a nylon salt solution comprising metering dicarboxylic acid powder, based on weight, from a loss-in-weight feeder to a feeding conduit that transfers the dicarboxylic acid powder into a disperser, such as an in-line disperser or a vessel with a disperser head, feeding a first feed stream of diamine to the disperser to form a dispersion comprising between 32 wt. % and 46 wt. % dicarboxylic acid, between 11 wt. % and 15 wt. % diamine, and between 39 wt. % and 57 wt. % water, heating the dispersion at temperature between 50° C. and 60° C. to form a PBA solution, introducing the PBA solution and a second feed stream of diamine to a continuous stirred tank reactor to form the nylon salt solution, continuously withdrawing the nylon salt solution from the continuous stirred tank reactor directly into a storage tank, wherein the nylon salt solution comprises between 50 and 65 wt. % salt concentration and comprises a dicarboxylic acid/diamine salt having a target pH, and controlling the feed rate variability of the dicarboxylic acid powder so that the target pH varies by ±0.04 pH. Preferably, the process of the present invention allows for the PBA solution to have low variability in adipic acid concentration of less than ±5%, e.g, preferably less than ±2%, less than ±1%, or less than ±0.5%.

### Weight-Based AA Feeder

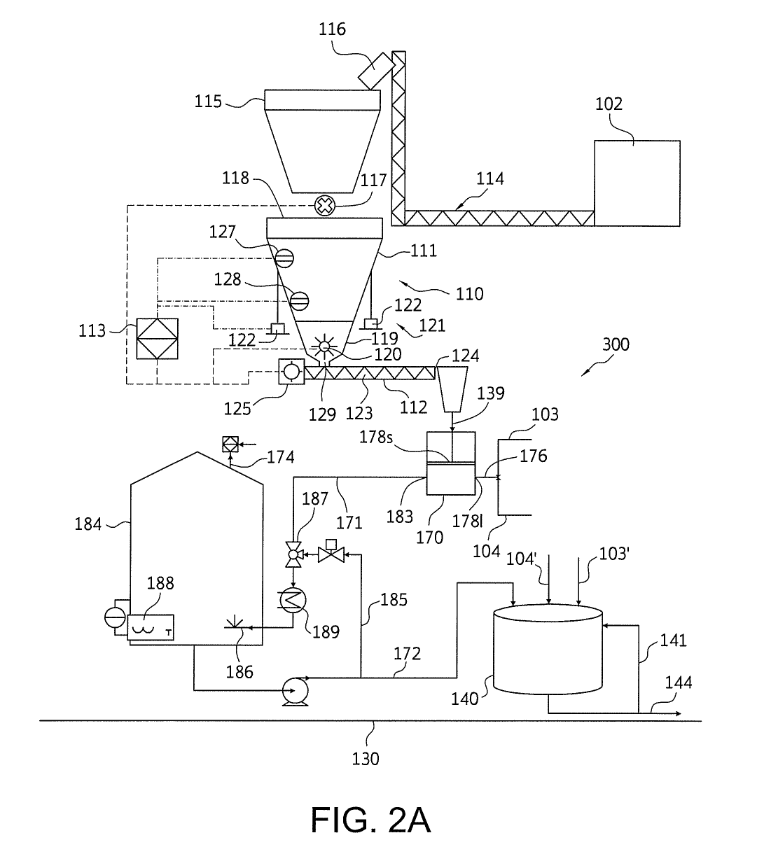

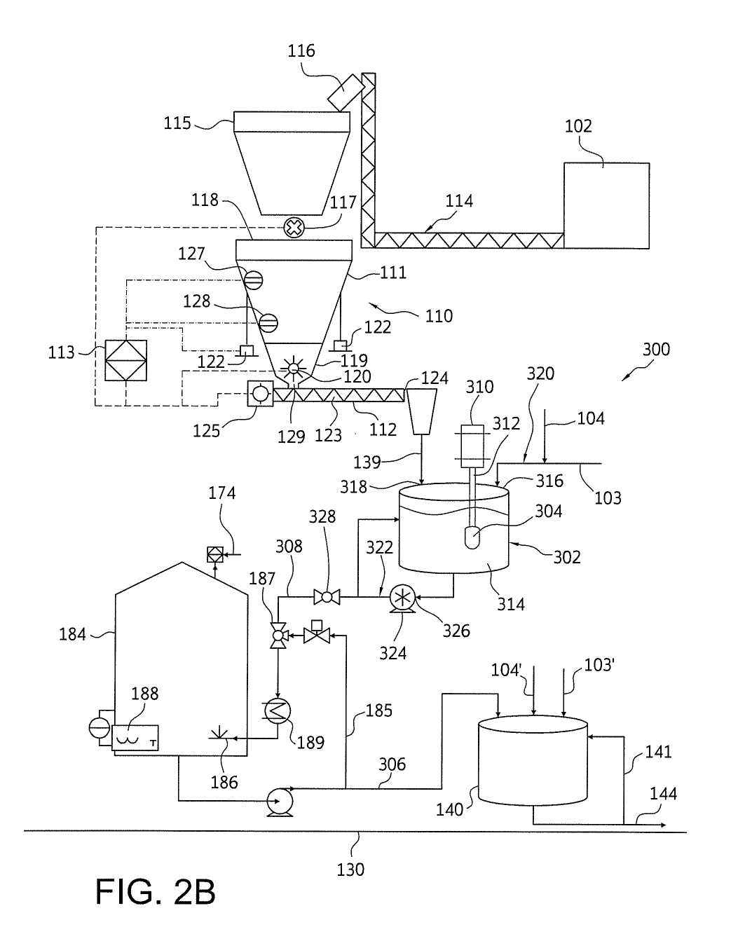

FIGS. 2A and 2B provide further details of producing PBA solution 172 that is enriched in AA. As shown in FIG. 2A, AA powder 102 is fed to in-line disperser 170 using a loss-in-weight feeder 110. As shown in FIG. 2B, AA powder 102 is fed to vessel 302 using a loss-in-weight feeder 110. Loss-in-weight feeder 110 meters AA powder 102 to produce an AA powder feed stream 139 having a low variability feed rate and is able to account for changes in density of AA powder 102 during the feeding process. As indicated above, AA powder 102 may vary greatly in bulk density and flow characteristics, leading to the introduction of imbalances in the molar ratio and producing a non-uniform pH for the nylon salt solution. The present invention is advantageous over a volumetric feeder or other types of feeders that cannot achieve a low variability feed rate of AA powder. For purposes of the present invention, a low variability feed rate of AA powder is within ±5% of the target feed rate of AA powder, e.g., within ±3%, within ±2%, or within ±1%. For purposes of the present invention, variability of feed rate refers to the average variation over a continuous operation. Because of the low variability of the AA powder feed rate, the feed rate of the AA is stable and predictable. The low variability of the AA powder feed rate may allow formation of a PBA solution with a low variability in adipic acid concentration. A stable and predictable AA powder feed rate may also allow the diamine and water feed rates to be tailored to achieve a target pH and/or a target salt concentration using a single reactor. Additional reactors are not needed for blending or adjustment because of the low variability of the AA powder feed rate from a target feed rate.

In general, a loss-in-weight feeder 110 operates to load a hopper 111 during a replenishment phase and dispense the contents of hopper 111 during a feed phase. Preferably, this replenishment-feed phase cycle is sufficient to receive a feedback signal from loss-in-weight feeder 110 at least 50% of the time, e.g., preferably at least 67% of the time. In one embodiment, the replenishment phase may be less than 20% of the total cycle time (e.g., total time for the feed and replenishment phases), e.g., less than 10% of the total cycle time or less than 5% of the total cycle time. The replenishment phase and total cycle phase times may be dependent on production rates. During the feed phase, the contents of hopper 111 are dispensed into a feeding conduit 112 that transfers the AA powder into continuous stirred tank reactor 140 via line 139. In addition, during the replenishment phase, the AA remaining in hopper 111 may also be dispensed into feeding conduit 112 so that feeding conduit 112 receives a constant supply of AA powder. A controller 113 may be used to regulate loss-in-weight feeder 110. Controller 113 may be a distributed control system (DCS) or a programmable logic controller (PLC) that is capable of outputting a function in response to a received input. In one embodiment, there may be multiple controllers for various components of the system. For example, a PLC may be used to regulate the replenishment phase and a DCS may be used to control the feed rate through the feeding conduit 112 from a target speed set in the DCS.

In one embodiment, the present invention is directed to a continuous process for producing a nylon salt solution comprising: metering dicarboxylic acid powder, based on weight, from a loss-in-weight feeder to a feeding conduit that transfers the dicarboxylic acid powder into an in-line disperser; feeding a first feed stream of diamine to the in-line disperser to form a dispersion comprising between 32 wt. % and 46 wt. % dicarboxylic acid, between 11 wt. % and 15 wt. % diamine, and between 39 wt. % and 57 wt. % water; heating the dispersion at temperature between 50° C. and 60° C. to form a partially balanced acid solution; introducing the partially balanced acid solution and a second feed stream of diamine to a continuous stirred tank reactor to form the nylon salt solution; continuously withdrawing the nylon salt solution from the continuous stirred tank reactor directly into a storage tank, wherein the nylon salt solution comprises between 50 and 65 wt. % salt concentration and comprises a dicarboxylic acid/diamine salt having a target pH; and controlling the feed rate variability of the dicarboxylic acid powder so that the target pH varies by ±0.04 pH.

Conveyance system 114 loads AA powder 102 into a supply vessel 115. Conveyance system 114 may be a mechanical or pneumatic conveyance system to transfer adipic acid from bulk bags, lined bulk bags, lined box containers or hopper railcar unloading stations. Mechanical conveyance systems may include screws or drag chains. Pneumatic conveyance systems may include enclosed tubes to deliver AA powder 102 using pressurized air, vacuum air, or closed loop nitrogen to supply vessel 115. In some embodiments, conveyance system 114 may provide mechanical functions to break clumps of AA powder while loading supply vessel 115. Supply vessel 115 may have a cylinder, trapezoid, square or other suitable shape having an entry 116 at the top. Shapes having angled sides are useful to assist the flow of AA powder 102 out of supply vessel 115. The upper edge of supply vessel 115 may be less than 20 meters (m), e.g., preferably less than 15 m above a system floor elevation 130. System floor elevation 130 refers to the planar surface upon which the various equipment to produce the nylon salt solution rests and generally defines a planar surface through which no monomers pass. The system floor elevation may be above the inlets of the CSTR. Due to the lower height of supply vessel 115 relative to system floor elevation 130, less energy is needed to drive conveyance system 114 and load supply vessel 115.

Supply vessel 115 also has lower valve 117 that when closed, defines an internal cavity for holding AA powder 102. Lower valve 117 may be a rotary feeder, a screw feeder, a rotating flow device or a combination device comprising a feeder and a valve. Lower valve 117 may be kept closed when filling the internal cavity with AA powder 102. Lower valve 117 may be opened during the replenishment phase to convey AA powder 102 on a volumetric basis to hopper 111. AA powder 102 may be loaded into supply vessel 115 when lower valve is conveying AA powder to hopper 111. Lower valve 117 may comprise one or more flaps that form a seal when closed. In one embodiment, there may be a conveying belt (not shown) to transfer AA powder 102 from supply vessel 115 to hopper 111. In other embodiments, supply vessel 115 may transfer AA powder 102 by gravity. The loading of supply vessel 115 may be independent of the loading of hopper 111.

Supply vessel 115 may have capacity that is larger than hopper 111, preferably a capacity that is at least twice as large or at least three times as large. The capacity of supply vessel 115 should be sufficient to replenish the entire volume of hopper 111. AA powder 102 may be held in supply vessel 115 for longer period of time than hopper 111, and depending on the moisture concentration, AA powder 102 may form clumps. The clumps may be broken by a mechanical rotator or vibration (not shown) at the base of supply vessel 115.

The upper edge of hopper 111 may be less than 15 m, e.g., preferably less than 12 m above system floor elevation 130. Hopper 111 may have cylinder, trapezoid, square or other suitable shape having an entry 118 at the top. Preferably, the internal surfaces of hopper are steep to prevent bridging of AA powder. In one embodiment, the internal surfaces have an angle from 30° to 80°, e.g., from 40° to 65°. The internal surfaces may be U-shaped or V-shaped. Hopper 111 may also have a removable lid (not shown) with an aperture for the entry 118 and vent opening. Hopper 111 may be mounted to duct 119 that connects hopper 111 to feeding conduit 112. In one embodiment, hopper 111 has an equivalent volume to maintain desired production rates. For example, hopper 111 may have a capacity of at least 4 tonnes. Duct 119 has a maximum diameter that is smaller than the maximum diameter of hopper 111. As shown, duct 119 has a rotary feeder 120, or similar transfer device, for dispensing the contents of hopper 111 through an exit 129 into feeding conduit 112. Rotary feeder 120 may be operated in on/off mode or the rotation rate can be controlled as a function of the desired feed rate. In other embodiments, duct 119 may have no internal feeder mechanisms. Depending on the type of loss-in-weight feeder, rotary feeder 120 may be replaced by external massage paddles or vibrators that dispense the discharge from hopper 111 to feeding conduit 112. Exit 129 may have a mechanical means to break clumps of AA. In another embodiment, loss-in-weight feeder 110 may have a drier or dry gas purge (not shown) to remove moisture from the AA powder to prevent the AA powder from setting up in hopper 111 and forming clogs.

A weight measuring subsystem 121 is connected to hopper 111. Weight measuring subsystem 121 may comprise a plurality of sensors 122 that weigh hopper 111 and provide a signal indicative of the weight to controller 113. In some embodiments there may be three sensors or four sensors. Sensors 122 may be connected to an external side of hopper 111 and may be tared to account for the initial weight of hopper 111 and any other equipment connected to hopper 111. In another embodiment, sensors 122 may be positioned underneath hopper 111. Based on the signals from weight measuring subsystem 121, controller 113 controls the replenishment phase and feed phase. Controller 113 compares the weight measured at regular intervals to determine the weight of AA powder 102 dispensed to feeding conduit 112 over a period of time. Controller 113 may also control the speed of rotating auger 123, described below.

In other embodiments, weight measuring subsystem 121 may be positioned under hopper 111, duct 119, and feeding conduit 112 to measure the weight of material in these locations of loss-in-weight feeder 110.

Feeding conduit 112 is located underneath duct 119 and receives AA powder 102. In one embodiment, feeding conduit 112 may be mounted to duct 119. Feeding conduit 112 may extend substantially perpendicular to the plane of exit 129 of duct 119 or may extend at an angle between 0° and 45°, e.g., between 5° and 40°, from that plane and towards disperser 300. Feeding conduit 112 has at least one rotating auger 123 that conveys AA powder 102 through an open outlet 124 and into reactor 140. Rotating auger 123 is driven by motor 125 and may comprise an endless screw. A double screw configuration may also be used. Motor 125 drives rotating auger 123, at fixed or variable speed. In one embodiment, feeding conduit 112 transfers AA powder 102 at a low variability feed rate into disperser 300. The feed rate of AA may be adjusted depending on the desired production rates. This allows the establishment of a fixed AA feed rate and using the model described herein, the feed rates of the other solution components are then varied to achieve desired salt concentrations and/or pH targets. Controller 113 receives feedback signals from loss-in-weight feeder 110 and adjusts the speed of rotating auger 123. Controller 113 also adjusts the feed rate of feeding conduit 112 based on the signals from weight measuring subsystem 121. The command signals to rotating auger 123 affect the motor speed, either increasing, maintaining, or decreasing it, to achieve the set weight loss.

In other embodiments, feeding conduit 112 described herein may be any equivalent, controllable feeder type such as a belt feeder, compartment feeder, plate feeder, vibrating feeder, etc. Feeding conduit 112 may also comprise a vibration dampener (not shown). In addition, feeding conduit 112 may have one or more gas ports (not shown) for injecting nitrogen to remove oxygen.

Hopper 111 may also comprise a high level probe 127 and a low level probe 128. It should be understood that for purposes of convenience, one high and low level probe are shown, but there may be multiple probes. The probes may be used in conjunction with weight measuring subsystem 121. For purposes of the present invention, the probes may be point level indicators or capacitive proximity sensors. The locations of high level probe 127 and low level probe 128 may be adjusted within hopper 111. High level probe 127 is positioned near the top of hopper 111. When material in hopper 111 is detected by high level probe 127, the replenishment phase is completed and the feed phase begins. Conversely, low level probe 128 is positioned below high level probe 127 and closer to the bottom of hopper 111. The position of low level probe 128 may allow a sufficient remaining amount of AA powder 102 to be dispensed during the replenishment phase. When low level probe 128 detects no material in hopper 111 at its position, the replenishment phase begins. As stated above, the feeding may continue during the replenishment phase.

AA solids can be corrosive. Loss-in-weight feeder 110 may be constructed of corrosion-resistant material such as an austenitic stainless steel or, for example, 304, 304L, 316 and 316L, or other suitable corrosion-resistant material to provide an economically viable balance between equipment longevity and capital cost. Additionally, the corrosion-resistant material may prevent corrosion contamination of the product. Other corrosion-resistant materials preferably are more resistant to attack by AA than carbon steel. HMD in high concentrations, e.g., greater than 65%, is not corrosive to carbon steel, and therefore carbon steel may be used for storing concentrated HMD, whereas stainless steel may be used to store more dilute HMD concentrations.

Although one exemplary loss-in-weight feeder 110 is shown, other acceptable loss-in-weight feeders, may include Acrison Models 402/404, 403, 405, 406, and 407; Merrick Model 570; K-Tron Models KT20, T35, T60, T80, S60, S100, and S500; and Brabender FlexWall™ Plus and FlexWall™ Classic. Acceptable loss-in-weight feeders 110 should be capable of achieving a feed rate sufficient for continuous commercial operation. For example, the feed rate may be at least 500 Kg/hr, e.g., at least 1000 Kg/hr, at least 5,000 Kg/hr or at least 10,000 Kg/hr. Higher feed rates may also be used with embodiments of the present invention.

### Disperser

In dissolving the AA powder, the present invention creates a homogenous mixture as a PBA solution with HMD and water. Water is beneficial to help dissolve the AA, because HMD is insufficient in dissolve AA powder. Water is also beneficial to reduce the freeze point of the resulting mixture. Adipic acid has low solubility in water, and therefore requires much higher storage temperatures without the presence of HMD.AA powder may be dissolved using a disperser 300 such as an in-line disperser 170 as shown in FIG. 2A or a vessel 302 with a disperser head 304 as shown in FIG. 2B. For purposes of the present invention, disperser 300 creates sufficient shear to produce a homogenous mixture.

Substantially all of the AA required for the nylon salt solution is passed through line disperser 300, and thus no AA powder needs to be dissolved in continuous stirred tank reactor 140. Disperser 300 produces a dispersion that is enriched in AA and that can be pumped as a PBA solution 306 to continuous stirred tank reactor 140. Advantageously, this improves AA feed uniformity to the reactor 140 and significantly increases the in-process storage capacity of adipic acid. For example, with liquid PBA solution 306, AA powder 102 may be stored in bins (not shown) that are within 15 meters of floor elevation 130, e.g., more preferably within 10 meters. Thus, loading of the bins is more easily accomplished.

Heat is required to maintain AA powder dissolved in water as a liquid due to the low solubility of AA in water. In one embodiment, one or more heaters may be provided in a recirculation loop. The heat necessary to maintain as a liquid may vary with water concentration. The present invention uses HMD and water to further assist dissolving AA and to form PBA solution 306 comprising a mixture that may be stored at a low temperature. Advantageously, the lower temperature of the mixture reduces the additional energy generally used to prevent slurry formation. In one embodiment, PBA solution 306 may be maintained at a temperature between 50° C. and 60° C., e.g., between 55° C. and 60° C., as a homogeneous solution. The mixture may be a slurry for a finite period of time until the acid has had time to fully dissolve, at which time the mixture will become a clear, homogeneous solution. Conditions for composition and temperature are set so that the initial slurry does not remain a slurry, but rather converts to the clear homogeneous solution. AA dissolution time is dependent on such variables as mixing energy, temperature, etc.

Disperser 300 forms a homogenous dispersion having a composition that may vary and generally comprises between 32 wt. % and 46 wt. % AA, between 11 wt. % and 15 wt. % HMD, and between 39 wt. % and 57 wt. % water, and more preferably, between 40 wt. % and 46 wt. % AA, between 13 wt. % and 15 wt. % HMD, and between 41 wt. % and 47 wt. % water. In one embodiment, the weight of AA in the partially balanced acid solution is at least twice the weight of HMD in the partially balanced acid solution. In one embodiment, dispersion comprises between 25% and 50% of a balanced salt, e.g., hexamethylene diammonium adipate salt, and between 15% and 40% free adipic acid. The solids concentration of dispersion may be less than 60%. Solids concentration includes the balanced salt and free AA. Generally, the dispersion does not contain any free HMD and all of the HMD fed to in-line disperser is chemically combined in the balanced salt. PBA solution 306 has the same composition and solid concentration as dispersion.

### In-Line Disperser

In a first embodiment, disperser 300 comprises an in-line disperser 170 which preferably is single-pass disperser that may be operated as a batch or continuous mixer. In-line disperser 170 may also have one or more gas ports (not shown) for injecting nitrogen to remove oxygen. If a nitrogen blanket is used, the nitrogen charged to the gas port suitably contains less moisture than the air surrounding the process unit. For example, dry nitrogen can be used.

Water 103 and HMD 104 may be fed to in-line disperser 170 through a liquid inlet 178l and AA powder 102 metered by loss-in-weight feeder 110 through a solid inlet 178s. For purposes of the present invention, at least 80% of the water needed to form a nylon salt solution with desired salt concentration between 50 and 65% may be introduced directly into in-line disperser 170, and more preferably at least 90% of the water needed. Generally, additional water may be added to vent condenser 131 or continuously stirred tank reactor 140 as a trim feed 103′, e.g., a second portion of water, to achieve the desired salt concentration. HMD 104 fed to in-line disperser 170 may be between 10% and 60% of the HMD needed to form the nylon salt solution, e.g., between 25% and 45% of the HMD needed. HMD 104 fed to in-line disperser 170 may be anhydrous or may contain between 0 wt. % and 20 wt. % water. The temperature of the HMD 104 fed to in-line disperser 170 may be sufficient to prevent solidification of HMD and is typically greater than 40° C., e.g. or greater than 45° C. The water may be added at ambient temperature to form dilute HMD solution 176 having a temperature of greater than 40° C., e.g. or greater than 45° C. In one embodiment, dilute HMD solution 176 comprises between 15 wt. % and 30 wt. % HMD, and from 70 wt. % to 85 wt. % water, more preferably between 20 wt. % and 30 wt. % HMD and from 70 wt. % to 80 wt. % water, may be fed to in-line disperser 170.

In one embodiment, the AA powder is dissolved in in-line disperser 170 in the presence of fresh HMD and water that are also fed to in-line disperser 170. Thus, a salt solution, either from reactor 140, storage tank 184 or storage tank 195 is not fed to in-line disperser 170 to dissolve the AA powder. Recirculation of the salt solution reduces the capacity of the process by as much as 50%.

AA, HMD, and water may be introduced into in-line disperser 170 in one or more successive charges when using a batch process. In one embodiment, there may two charges of monomers into in-line disperser 170. Each of the charges may be between 0.1 and 20 seconds, e.g., between 1 and 15 seconds. The first charge may comprise part of the AA, HMD, and water. In one embodiment, between 15% and 35% of the AA powder is introduced in the first charge, and more preferably between 20% and 30%. The temperature of solution in in-line disperser 170 rapidly rises with the first charge. The second charge comprises the remaining portion of AA. Additional charges of AA may also be added. As the subsequent charges are added and as further mixing occurs in in-line disperser 170, the temperature decreases due to the endothermic dissolution of AA in water. The process maintains the temperature of the solution in in-line disperser 170 above the initial temperature of the liquid HMD, e.g., above 45° C., to avoid forming a slurry or solidifying the solution. Therefore, the dispersion 171 is not a slurry.

In one exemplary embodiment, in-line disperser 170 comprises an internal cavity into which monomers are fed through one or more inlets 178 and a plurality of agitators to provide mechanical shear and to reduce the particle size of AA powder 102. As shown, in-line disperser 170 may have a powder inlet 178s and a liquid inlet 178l that both feed internal cavity. The plurality of agitators rotate around the internal cavity. In one embodiment, there are at least two different agitators having spaced apart blades. The monomers pass through the plurality of agitators into an external cavity and are forced out through the outlet 183. As shown in FIG. 2A outlet 183 enters a storage tank 184 through a recirculation loop 185. As shown, the recycle line returns to a lower portion of the storage tank through one or more internal jet mixers 186, such as eductors. In one embodiment, internal jet mixers 186 may be located between 0.3 and 1.5 meters from the bottom of storage tank 184, preferably between 0.5 and 1 meter. One or more jet mixers 186 may be used to blend or mix dispersion 171 into storage tank 184. PBA solution 306 may be taken from recirculation loop 185 as needed and fed to continuous stirred tank reactor 140. For purposes of the present invention, the liquid feed through liquid inlet 178l may be above atmospheric pressure when entering in-line disperser 170 and creates a low pressure zone (sub atmospheric) that creates suction for the solid feed through solid inlet 178s. Acceptable in-line dispersers may include QUADRO YTRON™, GERICKE® mixers, YSTRAL® mixers, and SILVERSON® high shear mixers.

In some embodiments, in-line disperser 170 may have a pressure differential of less than 200 kPa, e.g., of less than 170 kPa or less than 100 kPa. The use of the disperser discharge dispersion 171 as the motive flow for jet mixer 186 in storage tank 184 may require a higher pressure between 175 and 350 kPa. To increase the pressure of the disperser discharge through outlet 183, there may be an external eductor 187 at the convergence of the disperser discharge dispersion 171 and recirculation loop 185. Recirculation loop 185 serves as the motive flow for the external eductor to give dispersion 171 a pressure boost. In another embodiment, a booster pump (not shown) may be used in lieu of the external eductor 187 along the disperser discharge to storage tank 184.

In one embodiment, recirculation loop 185 does not have any analyzer directly measure the pH or salt concentration of PBA solution 306. A mass flow meter may be used in some embodiments to measure density and temperature and infer pH. Thus, PBA solution 306 is not adjusted by adding monomers in response to pH measurements from liquid in storage tank 184. Due to the enriched AA, PBA solution 172 is more acidic and less sensitive to compositional differences than the nylon salt solution as described below. Providing a stable feed of AA powder with low variation allows sufficient control of the PBA solution without the need to monitor and control the contents of storage tank 184. A supplemental pH measurement may be used in some optional embodiments.

Vessel with Disperser Head

In a second embodiment, disperser 300 may comprise a vessel 302 with a disperser head 304 as shown in FIG. 2B. In one embodiment vessel 302 may be operated as a batch or continuous high shear mixer. Vessel 302 may be a mixing chamber, vat or tank, such as a continuous stirred tank reactor, that has one or more disperser heads 304. Preferably a disperser head provides improved circulation to form the dispersion as compared to rotor-stator mixers. Vessel 302 is advantageously used to disperse AA powder 102 into liquid HMD 104 and water 105.

Each disperser head 304 is connected to a motor 310 by shaft 312. Motor 310 speed may be adjustable as desired to create sufficient mixing of reactant mixture. Disperser head 304 may be affixed to shaft 312, and is maintained below the liquid level in vessel 302. In some embodiments, disperser head 304 may be removable from shaft 312 to allow for disconnection and/or to switch between disperser heads. A suitable disperser head is described in U.S. Pat. No. 5,407,271, the entire contents and discloses of which are hereby incorporated by reference. Reactants are added to the contents of vessel 302 to form a reactant mixture 314. Disperser head 304 provides high shear mixing to form the dispersion that comprises a homogenous mixture. The size and shape of disperser head 304 may vary. In one embodiment, reactant mixture 314 is drawn into an internal chamber within disperser head 304 and is mechanically ripped by impeller blades or teeth. The mechanical ripping may be provided on the top and bottom of disperser head 304. Drawing the reactants into the top and bottom of disperser head 304 may create high velocity counter current streams that converge in the internal chamber to create high turbulence and hydraulic shear. Centrifugal pressure forces the contents out through openings on the side of disperser head 304. The edges of the openings may be sharpened to provide further mechanical shear. The high velocity discharge combines with the reactant mixture 314 to provide additional hydraulic shear and circulation.

In one optional embodiment, shaft 312 may comprise one or more mixing blades (not shown) to further assist in mixing.

Vessel 302 may also have one or more gas ports (not shown) for injecting nitrogen to remove oxygen. If a nitrogen blanket is used, the nitrogen charged to the gas port suitably contains less moisture than the air surrounding the process unit. For example, dry nitrogen can be used.

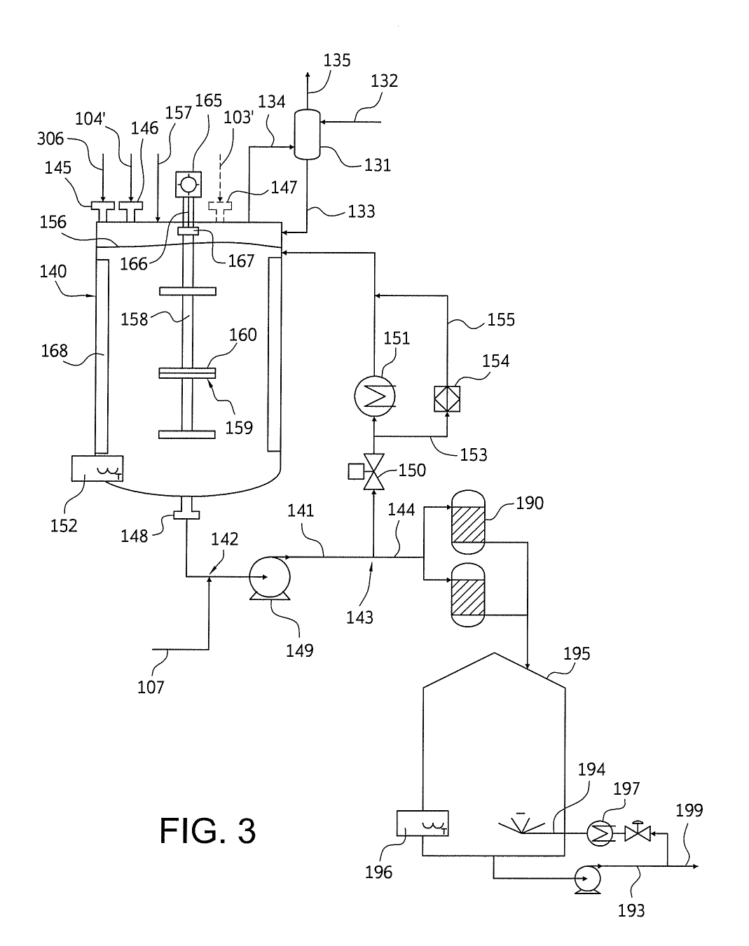

In one embodiment, water 103 and HMD 104 may be fed to vessel 302 through a liquid inlet 316 and AA powder 102 metered by loss-in-weight feeder 110 through a solid inlet 318. Liquid inlet 316 and solid inlet 318 may be on the top of vessel 302. For purposes of the present invention, at least 80% of the water needed to form a nylon salt solution with desired salt concentration between 50 and 65% may be introduced directly into vessel 302, and more preferably at least 90% of the water needed. Generally, additional water may be added to reactor vent condenser 131 as shown in FIG. 3, or to reactor 140 as a trim feed 103′, e.g., a second portion of water, to achieve the desired salt concentration. HMD 104 fed to vessel 302 may be between 10% and 60% of the HMD needed to form the nylon salt solution, e.g., between 25% and 45% of the HMD needed. HMD 104 fed to vessel 302 may be anhydrous or may contain between 0 wt. % and 20 wt. % water. The temperature of the HMD 104 fed to vessel 302 may be sufficient to prevent solidification of HMD and is typically greater than 45° C., e.g. or greater than 40° C. The water may be added at ambient temperature to form dilute HMD solution 320 having a temperature of less than 45° C., e.g. or less than 40° C. In one embodiment, dilute HMD solution 320 comprises between 15 wt. % and 30 wt. % HMD, and between 70 wt. % and 85 wt. % water, more preferably between 20 wt. % and 30 wt. % HMD and between 70 wt. % and 80 wt. % water, may be fed to vessel 302.

In one embodiment, the AA powder is dispersed and dissolved in vessel 302 in the presence of fresh HMD and water due to the high shear mixing created by disperser head 304. Thus, a salt solution, either from reactor 140, storage tank 184 or storage tank 195 is not fed to vessel 302 to dissolve AA powder. Recirculation of the salt solution reduces the capacity of the process by as much as 50%.

In one embodiment, reactant mixture 314 may be continuously recirculated in loop 322. To further assist in dispersing and milling the AA powder, loop 322 may comprise an in-line mixer 324 for continuous processing of dispersion 308. In-line mixer 324 may be a high shear mixer and disperser. Reactant mixture 314 enters through an inlet 326 and is sheared by internal mechanical stators, impellers and blades to form the homogenous mixture. There may be multiple mechanical stages for creating the shearing action. For examples, reactant mixture 314 may pass through rotating blades and into a stator when the reactant mixture 314 is sheared as it passes through slots in the stator. Dispersion 308 may be forwarded to storage tank 184 by means of a level control valve 328. A portion of dispersion 308 may also be recirculated to vessel 302. In some embodiments, external heating or cooling in loop 322 may be used to regulate the temperature of vessel 302. Preferably to maintain a homogeneous solution free of suspended crystals, the temperature of the contents in loop 322 and vessel 302 should be above 50° C., e.g., from 50° C. to 60° C. or from 55° C. to 60° C. The temperature may be controlled by regulating the flow through loop 322 and/or by regulating steam or hot water to a recirculation loop heater.

In one embodiment, the liquid feed through liquid inlet 326 may be above atmospheric pressure when entering in-line mixer 324 and creates a low pressure zone (sub atmospheric) that creates suction therethrough. Although one in-line mixer 324 is shown in FIG. 2B, in some embodiments, there may be multiple in-line mixers arranged in parallel or series. Suitable commercially available in-line mixers may include Admix DYNASHEAR™ mixers, QUADRO YTRON™ mixers, INOXPA™ in-line mixer ME 4100, IKA™ Works mixers, GERICKE™ mixers, YSTRAL™ mixers, and SILVERSON™ mixers.

In some embodiments, a pump, such as a centrifugal or positive displacement pump, may be used in loop 322 to provide further mixing of reactant mixture 314. The pump (not shown) may be used in addition to in-line mixer 324 or may be used independently of in-line mixer 324 if sufficient mixing is obtained with disperser head 304.

In one embodiment, recirculation loop 322 is not connected to any analyzers to directly measure the pH or salt concentration of dispersion 308. A mass flow meter may be used in some embodiments to measure density and temperature and infer pH. In addition, no monomers are added through recirculation loop 322 to adjust pH in response to a pH measurement.

As shown in FIG. 2B dispersion 308 enters a storage tank 184 through a recirculation loop 185. As shown, the recirculation loop returns to a lower portion of the storage tank through one or more internal jet mixers 186, such as eductors. In one embodiment, internal jet mixers 186 may be located between 0.3 and 1.5 meters from the bottom of storage tank 184, preferably between 0.5 and 1 meter. One or more jet mixers 186 may be used to blend or mix the dispersion into storage tank 184. PBA solution 306 may be taken from recirculation loop 185 as needed and fed to continuous stirred tank reactor 140.

In some embodiments, in-line mixer 324 may have a pressure differential of less than 200 kPa, e.g., of less than 170 kPa or less than 100 kPa. The use of dispersion 308 as the motive flow for jet mixer 186 in storage tank 184 may require a higher pressure between 175 and 350 kPa. To increase the pressure of dispersion 308 through level control valve 328, there may be an external eductor 187 at the convergence of dispersion 308 and recirculation loop 185. Recirculation loop 185 serves as the motive flow for the external eductor to give dispersion 308 a pressure boost. In another embodiment, a booster pump (not shown) may be used in lieu of the jet mixer along the disperser discharge to storage tank 184.

In one embodiment, recirculation loop 185 is not connected to any direct analyzers to measure or sample the stored dispersion, i.e. PBA solution 306. Due to the enriched AA, PBA solution 306 is more acidic and less sensitive to compositional differences than the nylon salt solution as described below. Thus, PBA solution 306 is not adjusted in response to pH measurements from liquid in storage tank 184. Providing a stable feed of AA powder with low variation allows sufficient control of the PBA solution without the need to monitor and control the contents of storage tank 184.

### Disperser Storage Tank

As shown in FIGS. 2A and 2B, storage tank 184 for the dispersion may have a capacity for up to a 5-day inventory of PBA solution, and more preferably up to a 3-day inventory. Although one storage tank 184 is shown, it is understood that multiple storage tanks may be used to hold the sufficient inventory. This allows disperser 300 to operate independently to dissolve adipic acid and the resulting dispersion may be stored prior to forming the nylon salt solution. Storage tank 184 may be maintained under an inert atmosphere, such as a nitrogen atmosphere, at atmospheric pressure or slightly above atmospheric pressure. Storage tank 184 may have a vent 174 for removing off-gas.

Storage tank 184 may be maintained at a temperature between 50° C. and 60° C., preferably between 55° C. and 60° C. Advantageously, the lower temperatures for storage may improve operating efficiencies, reduce salt degradation and reduce energy consumption. For example, feeding PBA solution directly, with no storage, to continuous stirred tank reactor 140 may provide for a 2 to 8 hour inventory, while feeding PBA solution from storage tank 184, which may hold 3 to 5 days of inventory, is an advantage of the present invention. This reduces the potential for interruptions to continuous stirred tank reactor 140 due to loss of the PBA solution feed. There may be an internal heater 188 in storage tank 184. In addition, recirculation loop 185 may have one or more heaters 189 for supplying heat to storage tank 184. Steam or hot water flow rates may be adjusted to either the internal heater 188 or one or more heaters 189 to maintain the desired temperature of storage tank 184.

In one embodiment, the pH of PBA solution 306 in storage tank 184 is not directly measured or adjusted by adding monomers to PBA solution 306 and/or storage tank 184. In one embodiment, the pH of the PBA solution 306 does not need to be measured prior to introducing the PBA solution 306 into continuous stirred tank reactor 140. A supplemental pH measurement may be used in some optional embodiments.

As described below, using a vessel 302 to form a PBA solution not only reduces the number of salt reactors in series, but the PBA solution advantageously increases in-process adipic inventory and carries of a portion of target salt inventory as semi-finished inventory at significantly lower temperature for reduced salt degradation, improves AA feed uniformity to continuous stirred tank reactor 140, and eliminates separate batch PBA facilities as a polymerizer ends modification additive.

### Reactor

In one embodiment of the present invention, the nylon salt solution is prepared from PBA solution 306 in a single continuous stirred tank reactor 140 as shown in FIG. 3. Continuous stirred tank reactor 140 creates sufficient turbulent flow for producing a homogeneous nylon salt solution. For purposes of the present invention, “a continuous stirred tank reactor” refers to one reactor and does not include multiple reactors. In addition, the single reactor does not include vessel 302. The present invention is able to achieve the uniform nylon salt solution in a single vessel and does not require multiple cascading vessels as used in conventional processes. Suitable continuous stirred tank reactors are a single vessel reactor such as a non-cascading reactor. Advantageously, this reduces the capital investment in producing a nylon salt solution on a commercial scale. When used in combination with the loss-in-weight feeder described herein, the continuous stirred tank reactor is able to achieve a uniform nylon salt solution that achieves the target pH and target salt concentrations.

The nylon salt solution is withdrawn from reactor 140 and is directly transferred to storage tank 195. No subsequent introduction of monomers, either AA or HMD, are introduced into the nylon salt solution between withdrawal from continuous stirred tank reactor 140 and entry into storage tank 195. More specifically, the nylon salt solution is withdrawn in conduit 144 from recirculation loop 141 and no monomers are added into conduit 144. In one aspect, conduit 144 does not have inlets for introduction of additional monomers that may include dicarboxylic acids and/or diamines. Thus, the pH of the nylon salt solution is not further adjusted by introducing additional monomers to the conduit, and in particular is not adjusted by adding additional HMD. There may be additional mixing and filtration of the nylon salt solution as needed, but the monomers are only fed to the single continuous stirred tank reactor as described herein. Thus the disclosed process avoids the need for the sequence of multiple vessels and successive steps of pH measurement and adjustment previously believed to be needed to maintain a stable stoichiometric balance between AA and HMD for making nylon 6,6.

Continuous stirred tank reactor 140 may have a height to diameter ratio between 1 and 6, e.g., between 2 and 5. Reactor 140 may be constructed of a material selected from the group consisting of Hastelloy C, aluminum, and an austenitic stainless steel such as 304, 304L, 316 and 316L, or other suitable corrosion-resistant material to provide an economically viable balance between equipment longevity and capital cost. The selection of the material may be made by considering temperature in continuous stirred tank reactor 140. The residence time in continuous stirred tank reactor 140 may vary depending on the size and feed rates, and is generally less than 45 minutes, e.g., less than 25 minutes. The liquid is withdrawn in a lower outlet 148 into recirculation loop 141 and a nylon salt solution is withdrawn in conduit 144.

In general, a suitable continuous stirred tank reactor comprises at least one monomer inlet for introducing HMD, and/or water and an inlet for introducing the PBA solution. The inlets are directed to an upper portion of the reactor. In some embodiments, the monomers drop into the liquid. In other embodiments, diptubes may be used to feed the monomers at the liquid level. There may be multiple inlets for introducing each component in the reaction medium. An exemplary reactor 140 is shown in FIG. 3. When a PBA solution is used, it is preferred that the balance of AA for the nylon salt solution is fed to disperser 300 to create the PBA solution 306. Thus, continuous stirred tank reactor 140 preferably has a PBA inlet 145 and HMD inlet 146 and no solids are introduced into continuous stirred tank reactor 140. The HMD may be introduced as pure HMD 104′ or as an aqueous solution comprising between 20 wt. % and 100 wt. % HMD, e.g., between 65 wt. % and 100 wt. %, and between 0 wt. % and 80 wt. % water, e.g., between 0 wt. % and 20 wt. %. HMD 104′ fed to continuous stirred tank reactor 140 may be between 20% and 70% of the HMD needed to form the nylon salt solution, e.g., between 30% and 55% of the HMD needed. HMD 104′ may be introduced through inlet 146 that is adjacent to inlet 145 of PBA solution 306. Because the tolerance for salt concentration is less tight than pH, water may be introduced at a number of locations, such as through inlets 145 and/or 146 and/or through pump 149, described herein. Optionally there may be an inlet 147 for separately introducing the water. Water may also be introduced through a reactor recovery column 131. In some aspects, recovery column 131 is a vent condenser. Because the majority of water is introduced with PBA solution 306, a small amount of water may be needed to achieve the desired salt concentration.

The liquid in reactor 140 is continuously withdrawn and passes through recirculation loop 141. Recirculation loop 141 may comprise one or more pumps 149. Recirculation loop 141 may also comprise a temperature control device, e.g., coils, a jacket, or a device comprising a heat exchanger, temperature measurement device and controller. The temperature control device controls the temperature of the nylon salt solution in recirculation loop 141 to prevent boiling or slurrying of the nylon salt solution. When additional HMD, e.g., trim HMD, is introduced via line 107, it is preferred to introduce HMD upstream of one or more pumps 149 at junction 142 and upstream of any pH or salt concentration analyzers. As discussed further herein, trim HMD 107 may contain between 1% and 20% of the HMD needed to form the nylon salt solution, e.g., between 1% and 10% of the HMD needed. Junction 142 may be a feed port into recirculation loop 141. In addition to recirculating the liquid, the pumps 149 also function as secondary mixers. The pumps may function to both introduce the trim HMD into recirculation loop 141 and to mix the trim HMD with the liquid withdrawn from the reactor. The pumps may be selected from the group consisting of vane pumps, piston pumps, flexible member pumps, lobe pumps, gear pumps, circumferential piston pumps, and screw pumps. In some embodiments, pumps 149 are located at junction 142. In other embodiments, as shown, pumps 149 are located downstream of junction 142 but before junction 143. It is preferred that the secondary mixing occur after the addition of all the HMD, including the trim HMD through line 107, and prior to any analyzing or withdrawing into storage tank 195. In alternative embodiments, the one or more static mixers (not shown) may be placed downstream of pumps 149 in recirculation loop 141. Exemplary static mixers are further described in Perry, Robert H., and Don W. Green. Perry's Chemical Engineers' Handbook. 7th ed. New York: McGraw-Hill, 1997: 18-25 to 18-34, hereby incorporated by reference.

At junction 143, the nylon salt solution may be withdrawn in conduit 144. The residence time in conduit 144 may varying depending on the location of storage tank 195 and filters 190, and is generally less than 600 seconds, e.g., less than 400 seconds. In one embodiment, valve 150 may be operated to control the pressure of the nylon salt solution. Although one valve is shown, it should be understood that additional valves may be used in recirculation loop 141. No monomers, e.g., AA or HMD, are introduced downstream of junction 143 or into conduit 144. In addition, no monomers are introduced into storage tank 195 under normal operating conditions.

Recirculation loop 141 may also comprise a heat exchanger 151 for regulating the temperature of the liquid in reactor 140. The temperature may be regulated by using a temperature controller (not shown), either in reactor 140 or at a continuous stirred tank reactor 140 outlet (not shown). The temperature of the liquid may be regulated using an internal heat exchanger, such as a coil or a jacketed reactor (not shown). Heat exchanger 151 may be supplied with cooling water that is maintained above the freezing point of the salt for a given concentration. In one embodiment, the heat exchanger may be an indirect shell and tube exchanger, a spiral or plate and frame heat exchanger, or a reboiler for heat recovery from reactor 140. The temperature in reactor 140 is maintained within a range between 60° C. and 110° C. to prevent slurry formation and crystal formation. As the water concentration increases, the temperature to maintain a solution decreases. In addition, the temperature in reactor 140 is maintained at low temperature to deter oxidation of HMD. A nitrogen blanket may also be provided to deter oxidation of HMD.

As shown in FIG. 3, in one embodiment, reactor 140 has an internal coil 152 into which a coolant may be fed to regulate the temperature of the reactor within a temperature that is between 60° C. and 110° C. In another embodiment, reactor 140 may also be jacketed with a coolant (not shown). The internal coil may also regulate the temperature by recovering heat produced by the reaction.

In addition to a temperature controller, reactor 140 may also have an atmospheric vent with a vent condenser to maintain atmospheric pressure within reactor 140. The pressure controller may have internal and/or external pressure sensors.

In one embodiment, there may also be a sample line 153 for measuring the pH and/or salt concentration of the nylon salt. Sample line 153 may be in fluid communication with recirculation loop 141 and preferably receives a fixed flow therethrough to minimize the influence of flow on the analyzers. In one aspect, sample line 153 may withdraw less than 1% of the nylon salt solution in recirculation loop 141, and more preferably less than 0.5%. There may be one or more analyzers 154 in sample line 153. In some embodiments, sample line 153 may comprise a filter (not shown). In another embodiment, sample line 153 may contain suitable heating or cooling devices such as heat exchangers to control the temperature of the sample stream. Similarly, sample line 153 may include a water charge line (not shown) for adding water to the sample stream to adjust concentration. If water is added to the sample stream, the water can be deionized water. The water fed through sample line 153 is calculated to maintain the target salt concentration and the other feeds of water may be adjusted. Analyzers 154 may include on-line analyzers for real-time measurement. Depending on the type of sampling, the tested portion may be returned to reactor 140 via line 155 or discharged. Sample line 153 may be returned through recirculation loop 141. Alternatively, sample line 153 is returned at a separate location into reactor 140.

Continuous stirred tank reactor 140 maintains liquid level 156 that is at least 50% full, e.g., at least 60% full. The liquid level is selected so that it is sufficient to submerge the blades of the CSTR and thus prevent foaming of the nylon salt solution. Nitrogen or another inert gas may be introduced into head space above liquid level 156 through a gas port 157.

Agitator shaft 158 may have one or more impellers 159 such as mixing paddles, helical ribbons, anchors, screw-types, propellers, and/or turbines. Axial flow impellers are preferred for mixing AA and HMD because these impellers tend to prevent the solid particles from settling at the bottom of reactor 140. In other embodiments, the impeller may be a flat-blade radial turbine having multiple blades equally spaced around a disk. In total agitator shaft 158 may have between 2 and 10 impellers, e.g., between 2 and 4 impellers. The blades 160 on impeller 159 may be straight, curved, concave, convex, angled, or pitched. The number of blades 160 may vary between 2 and 20, e.g., between 2 and 10. If needed, blades 160 may also have stabilizers (not shown) or scrapers (not shown).

Agitator shaft 158 may have one or more impellers 159 such as mixing paddles, helical ribbons, anchors, screw-types, propellers, and/or turbines. Axial flow impellers are preferred for mixing AA and HMD because these impellers tend to prevent the solid particles from settling at the bottom of reactor 140. In other embodiments, the impeller may be a flat-blade radial turbine having multiple blades equally spaced around a disk. In total agitator shaft 158 may have between 2 and 10 impellers, e.g., between 2 and 4 impellers. Blades 160 on impeller 159 may be straight, curved, concave, convex, angled, or pitched. The number of blades 160 may vary between 2 and 20, e.g., between 2 and 10. If needed, blades 160 may also have stabilizers (not shown) or scrapers (not shown).

In one exemplary embodiment, the agitator shaft may be a triple-pitch turbine assembly. In this type of assembly, agitator shaft 159 comprises at least one upper pitch blade turbine (not shown) and at least one lower pitch blade turbine (not shown). In the triple-pitch turbine assembly, the angled faces of the upper pitch blade turbine are preferably offset from the angled faces of the lower pitch blade turbine.

Multiple agitator shafts with different types of impellers, such as spirals and anchors, may also be used. Also, side mounted agitator shafts may be used, in particular those with marine propellers.

Agitator shaft 158 is driven by an external motor 165 that may mix the liquid between 50 and 500 rpm, e.g., between 50 and 300 rpm. Agitator shaft 158 may be removably mounted to the motor drive shaft 166 at connector 167. The speed of the motion may vary, but generally the speed should be sufficient to maintain the entire surface area of solid particles in contact with the liquid phase ensuring maximum availability of the interfacial area for mass transfer in a solid-liquid.

Reactor 140 may also comprise one or more baffles 168 for mixing and to prevent formation of dead zones. The number of baffles 168 may vary between 2 and 20, e.g., between 2 and 10, and are evenly spaced around the perimeter of reactor 140. Baffles 168 may be mounted on the internal wall of reactor 140. Generally, vertical baffles 168 are used, but curved baffles may also be used. Baffles 168 may extend above the liquid level 156 in reactor 140.

In one embodiment, reactor 140 comprises a vent for removing off-gas through line 135 and a recovery column 131 for returning condensable HMD to reactor 140. Water 132 may be fed to recovery column 131 and recovered in bottoms 133 of recovery column 131. The water fed at a minimum rate to maintain efficiency of recovery column 131 and that amount of water is calculated to maintain the target salt concentration and the other feeds of water may be adjusted. Vent gases 134 may be condensed to recover any water and monomer off gas and may be returned via line 133. Non-condensable gases, including nitrogen and air may be removed as an off-gas stream 135. When recovery column 131 is a vent condenser, recovery column 131 may be used to recover off-gas and remove non-condensable gases.

Although one exemplary continuous stirred tank reactor is shown, other acceptable continuous stirred tank reactor may be used.

### Nylon Salt Solution Storage

As shown in FIG. 3, as the nylon salt solution is formed, it is fed to a storage tank 195 where the nylon salt solution may be held until needed for polymerization. In some embodiments, storage tank 195 may comprise a recirculation loop 193 to circulate nylon salt solution. An internal jet mixer 194 may be used to maintain circulation within storage tank 195. In one embodiment, internal jet mixers 194 may be located between 0.3 and 1.5 meters from the bottom of storage tank 195, preferably between 0.5 and 1 meter. Additionally, in some embodiments, at least a portion of the nylon salt solution may be returned to reactor 140 to prevent process lines from freezing and/or to correct the nylon salt solution in the case of a system upset or a desired change in the target pH and/or target salt solution. Any unused nylon salt solution from the polymerization process 200 may also be returned to storage tank 195.

Storage tank 195 may be constructed of corrosion-resistant material such as an austenitic stainless steel, for example, 304, 304L, 316 and 316L, or other suitable corrosion-resistant material to provide an economically viable balance between equipment longevity and capital cost. Storage tank 195 may comprise one or more storage tanks, depending on the storage tank size and volume of nylon salt solution to be stored. In some embodiments, the nylon salt solution is stored in at least two storage tanks, e.g., at least three storage tanks, at least four storage tanks, or at least five storage tanks. Storage tank 195 may be maintained at a temperature above the solution freezing point, such as at a temperature between 60° C. and 110° C. For nylon salt solutions having a salt concentration between 60 wt. % and 65 wt. %, the temperature may be maintained between 100° C. and 110° C. There may be an internal heater 196 in the storage tank. In addition, the recirculation loop may have one or more heaters 197 for supplying heat to the storage tank. For example, the storage tank may have a capacity for up to a 5-day inventory of nylon salt solution, and more preferably up to a 3-day inventory. The storage tank may be maintained under a nitrogen atmosphere at atmospheric pressure or slightly above atmospheric pressure.

In some embodiments, before entering storage tank 195, the nylon salt solution may be filtered to remove impurities. The nylon salt solution may be filtered through at least one filter 190, e.g., at least two filters or at least three filters. The filters 190 may be arranged in series or in parallel. Suitable filters may include membrane filters comprising polypropylene, cellulose, cotton and/or fiberglass. In some embodiments, the filters may have pore sizes between 1 and 20 microns, e.g., between 2 and 10 microns. The filter may also be an ultrafiltration filter, a microfiltration unit, a nanofiltration filter, or an activated carbon filter.

### Trim HMD

As indicated in the above description, an equivalent amount HMD needed to form the nylon salt is introduced in different portions in at least three locations to form the nylon salt solution. The first portion is added to form the PBA solution. In addition, feed rate of the portion of HMD added to the disperser, i.e. in-line disperser or vessel with disperser head, may be fixed to provide the necessary amount of HMD to solubilize AA powder. The second and third portions are added to the CSTR to form the nylon salt solution. To allow the use of one continuous stirred tank reactor and to form a uniform nylon salt solution, HMD is not added once the nylon salt solution is withdrawn from the reactor into the conduit and subsequently to the storage tank. HMD is introduced into the disperser, continuous stirred tank reactor and to the recirculation loop of continuous stirred tank reactor as trim HMD. To allow for the use of a single continuous stirred tank reactor and to a uniform nylon salt solution, HMD is not added once the nylon salt solution is withdrawn from the reactor 140 into conduit 144, and subsequently to storage tank 195. Control of variance from the target specifications, e.g., target pH, may further be refined by including the trim HMD via line 107 as shown in FIG. 3 at junction 142. The trim HMD is generally the smallest portion of HMD added and is used as a fine tuning control for the pH of the nylon salt solution, due to the use of a smaller valve that has higher control of small changes in flow as compared to the main HMD feed. Adjusting the feed rate or flow rate of the main HMD is a less preferred method for controlling pH of the nylon salt solution because of the lag between the adjustment of the main HMD and the pH measurement. In addition, because the trim HMD is the smallest portion of HMD added to the CSTR, the trim HMD allows for more accurate adjustments of the pH of the nylon salt solution and the pH analyzer provides near instantaneous feedback. Trim HMD is added upstream of the pH measurement to reduce the delay in measuring the pH effect of adding the trim HMD. As the trim HMD is adjusted, the water feed rate may also be adjusted to control the concentration of solids in the nylon salt solution. Such adjustments may be set by controllers and may be monitored by refractometers in sample line 153, described herein.

Trim HMD 107 may be combined with the nylon salt solution before it enters conduit 144. Without being bound by theory, it is believed that trim HMD 107 may react with any remaining free AA in the nylon salt solution. Additionally, adding trim HMD 107 may be used to adjust the pH of the nylon salt solution as described above.

In one embodiment, the present invention is directed to metering PBA solution 306 into a continuous stirred tank reactor 140; separately introducing an aqueous solution comprising a first portion of HMD 104′ and water 103′ to continuous stirred tank reactor 140 to form a nylon salt solution; and introducing a second portion of HMD, e.g., trim HMD via line 107 to the nylon salt solution. First portion of HMD 104′ and water 103′ may be combined to form an aqueous HMD solution feed. Trim HMD 107 may be added to nylon salt solution in the recirculation loop 141 at junction 142. Trim HMD 107 is continuously fed to recirculation loop 141 at a feed rate that allows the flow of trim HMD 107 to be within the midrange flow through the valve, e.g., from 20 to 60%, from 40 to 50%, or about 50%. Midrange flow refers to maintaining a continuous flow through the valve to prevent a loss of control.

To achieve a target pH with low variability, the process involves providing a constant feed rate of AA powder 102 using loss-in-weight feeder 110 to form PBA solution 306, and adjusting the feed rates of HMD and water in response to process controls. Advantageously, high production rates may be achieved from a continuous process. When changing salt production rates, the HMD feed rate is adjusted proportionately as the AA feed rate is changed in discrete intervals. The feed rate of HMD may be adjusted by either changing the feed rate of the HMD fed to the reactor 140 or HMD fed as trim HMD. In one preferred embodiment, the feed rate of trim HMD 107 may be adjusted and the feed rate of HMD 104′ and/or the feed rate of the aqueous HMD solution feed may be constant for a given salt production rate. In alternative embodiments, the feed rate of trim HMD 107 may be set at a constant rate and the feed rate of HMD 104′ and/or the feed rate of the aqueous HMD solution feed may be adjusted, if necessary, to achieve the target pH and/or salt concentration. In still other embodiments, the feed rate of both HMD 104′ and trim HMD 107 and/or the feed rate of the aqueous HMD solution feed may be adjusted to achieve the target pH and/or salt concentration.

Trim HMD 107 may have the same HMD source as HMD 104′. HMD 104′ may comprise between 80% and 99% of the total HMD in the nylon salt solution, e.g., between 90% and 99%. Trim HMD 107 may comprise between 1 and 20% of the total HMD in the nylon salt solution, e.g., between 1% and 10%. The ratio of HMD 104′ and trim HMD 107 may be adjusted depending on target pH and target salt concentration. As discussed herein, the ratio of HMD 104′ and trim HMD 107 may be set by the model for the total HMD feed rate.

Trim HMD may have the same source as HMD for the disperser and continuous stirred tank reactor. The HMD may be supplied as neat HMD, e.g., comprising at least 99.5 wt. % HMD, e.g., 100% HMD and no water, or may be supplied in an aqueous solution comprising between 80 wt. % and 99.5 wt. % HMD. Trim HMD 107 is fed to the nylon salt solution as neat HMD or as an aqueous solution of HMD. When trim HMD 107 is an aqueous solution of HMD, the aqueous solution of trim HMD 107 may comprise between 50 wt. % and 99 wt. % HMD, e.g., between 60 wt. % and 95 wt. % HMD or between 70 wt. % and 90 wt. % HMD. As with the aqueous solution for HMD 104′, the amount of water may be adjusted based on the source of HMD and the desired salt concentration of the nylon salt solution. Advantageously, the HMD concentration of trim HMD 107 from 90 wt. % to 100 wt. % to enhance effect on pH control while minimizing the effect of trim HMD 107 in salt concentration control.

Trim HMD 107 is added to the nylon salt solution in the recirculation loop upstream of pumps 149 and sample line 153. The pH of the nylon salt solution in recirculation loop 141 may be measured in sample line 153 using analyzer 154, after the second portion of HMD 107 has been added. This allows a small delay between adjusting pH through the feed rate of trim HMD 107 and pH measurement. No additional AA is added to recirculation loop 141. No HMD, other than trim HMD 107 is added to recirculation loop 141. Second portion of HMD 107 is added upstream of pH measurement to allow for pH measurement that includes the second portion of HMD 107.

Unlike prior process shown in U.S. Pub. No. 2010/0168375 and U.S. Pat. No. 4,233,234, trim HMD is not added after the pH measurement. Adding HMD after the pH measurement creates a large delay in measuring the effect of the added HMD on the pH because the added HMD must pass through the reactor before being measured. Thus, adding HMD in such a manner may undershoot or overshoot the target pH which causes these processes to operate inefficiently by constantly chasing the target pH. Advantageously, the present invention adds trim HMD upstream of the pH measurement so that the effect of the trim HMD is accounted for with little delay and avoids the problems of undershooting or overshooting the target pH. In addition, the present invention constantly feeds trim HMD 107 because the valve is maintained at a midrange flow.

### Process Controls

As described herein, in a continuous process for producing a polyamide salt solution, e.g., a nylon salt solution, in prior art processes, there may be variability in target specifications in the nylon salt solution, including pH and salt concentration. This variability in the target specifications may be caused, at least in part, by unpredictable and fluctuating AA powder feed rate. Such unpredictability and fluctuations make controlling the process difficult, because the process must be constantly monitored and adjusted downstream of the initial reactor prior to storage. Thus, a single reactor operating continuously could not efficiently account for the unpredictable and fluctuating AA powder feed rate. Conventionally, in order to account for this unpredictability and fluctuation, numerous reactors, mixers, and multiple reactant feed locations, in particular for adding HMD are used to produce the nylon salt solution with target specifications. Using one continuous stirred tank reactor according to the present invention removes the ability to adjust the nylon salt solution in numerous reactors. However, an improved process control may be achieved by leveling the AA powder feed rate variability, i.e. an AA powder feed rate that varies by less than ±5%, by using a loss-in-weight feeder to form a PBA solution and using the PBA solution as the source of AA to form the nylon salt solution. In one aspect, the present invention uses feed forward controls based on a model, with or without feedback to achieve a nylon salt solution with target pH and salt concentration.

### Feed Forward Controls

Prior to beginning the continuous process for producing the nylon salt solution, a reaction model may be prepared based on a desired nylon salt solution production rate. Based on this production rate, an AA powder feed rate is set, and then target pH and target salt concentration are set. The HMD feed rate and water feed rate are then stoichiometrically calculated to achieve the target pH and target salt concentration. The HMD feed rate includes the HMD to form the PBA solution, main HMD to the reactor, and trim HMD. The water feed rate includes all sources of water fed to the disperser and reactor 140. It is understood that a target pH reflects a target molar ratio of AA to HMD. In further embodiments, additional features may be added to the model, including but not limited to reaction temperature and reaction pressure. This model is used to set feed forward controls of feed rates for the HMD and/or water to the disperser and the continuous stirred tank reactor. In some embodiments, the model may also be used to set feed forward controls of the PBA solution to the continuous stirred tank reactor.

In some aspects, the model is prepared by inputting the feed rate of AA powder provided by the loss-in-weight feeder described herein. The model may also set the feed rate of the HMD to the disperser to achieve the desired mixture. For a given production rate, the feed rate of AA should be constant. The loss-in-weight feeder may contain discrete controls, as described herein, to produce an AA powder feed rate with low variability. The AA powder feed rate from the loss-in-weight feeder may be provided continuous, semi-continuously, or at discrete intervals, e.g., every 5 minutes, every 30 minutes, or hourly, to the model. In other aspects, because of the low variability of the AA powder feed rate, once the feed rate of the AA powder is set, the model may set an HMD feed rate and a water feed rate. These feed rates are set by the model to achieve a target pH and a target salt concentration.

The model may be dynamic and may be adjusted by feedback signals from on-line and off-line analyzers. For example, if a change in production rate, pH or salt concentration is desired, the model may be adjusted. The model may be stored in memory of a controller, such as a programmable logic controller (PLC) controller, distributed control system (DCS) controller or a proportional-integral-derivative (PID) controller. In one embodiment, a PID controller, with feedback signals, may be used to account for errors in the model calculations and flow measurements.

Feed forward controls, by themselves, have previously been impractical to form a nylon salt solution with low variability from target specifications due to the inability to accurately predict AA powder feed rate with the use of volumetric feeders. This is due, at least in part, to variation in AA powder feed rates caused by the use of volumetric feeders. Because of the variability of the AA powder feed, a model could not be generated to control the AA and HMD ratio. Consequently with feed forward control, these conventional processes could use feedback controls, thus requiring frequent adjustments, or would be a batch process. However, when the AA powder is metered on a weight basis to a disperser, feed forward controls are sufficient to continuously produce a nylon salt solution with low variability from target specifications.

Thus, in one embodiment, the present invention is directed to a process for controlling the production of a nylon salt solution, comprising generating a model for a target feed rate of AA powder to produce the PBA solution, and subsequently the nylon salt solution having a target salt concentration and/or a target pH. As indicated above, the target salt concentration may be a value selected from the range between 50 and 65 wt. %, e.g., between 60 and 65 wt. %. The target pH may be a value selected from the range between 7.200 and 7.900, e.g., between 7.400 and 7.700. The process may further comprise separately introducing HMD at a first feed rate and water at a second feed rate to the disperser, wherein the first and/or the second feed rate is based on the model for the PBA solution. The process may further comprise separately introducing the PBA solution at a third feed rate to the continuous stirred tank reactor, wherein the third feed rate is based on the model for the nylon salt solution. The process may further comprise separately introducing HMD at a fourth feed rate and water at a fifth feed rate to the continuous stirred tank reactor, wherein the fourth and/or the fifth feed rate is based on the model for the target feed rate of AA powder. The HMD and PBA solution react to form a nylon salt solution which may then be continuously withdrawn from the continuous stirred tank reactor directly into a storage tank. The nylon salt solution may then be stored for future polymerization reactions. Regardless of the target salt concentration or pH selected, the actual specifications of the nylon salt solution have a low variability from the target specification, such as less than 0.53% variability, e.g., less than 0.4%, less than 0.3% or less than 0.1%.

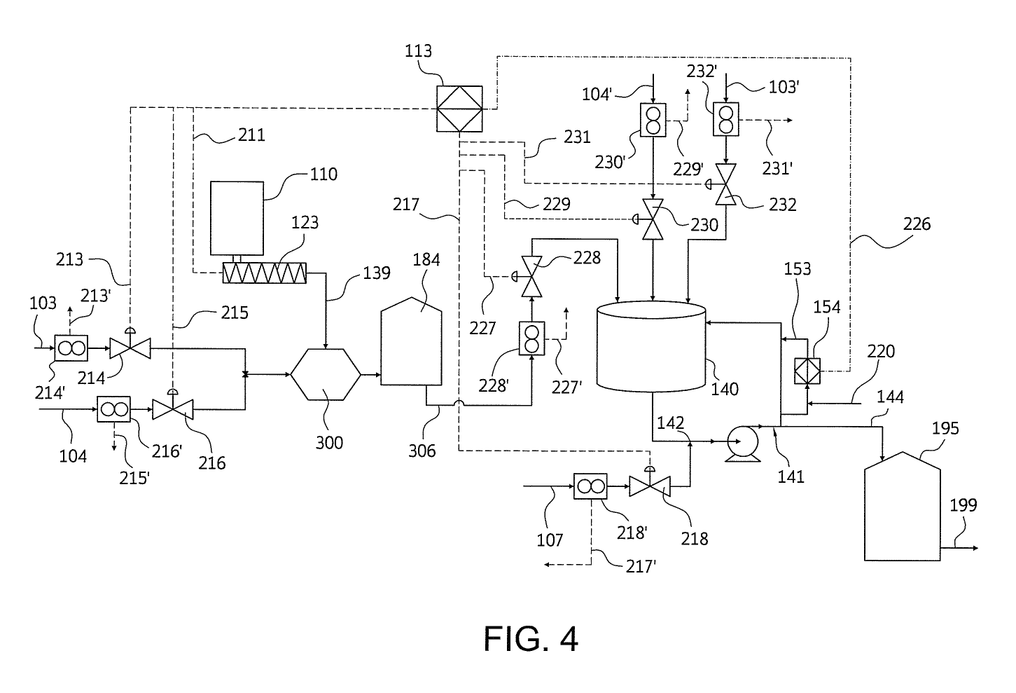

To further exemplify the process control schemes according to the present invention, a schematic diagram is shown in FIG. 4. For simplicity, FIG. 4 excludes various pumps, recirculation loops, and heaters. Several flow meters, such as coriolis mass flow meters, positive displacement flow meters, electromagnetic flow meters, and turbine flow meters, are shown in FIG. 4 for measuring flow through the system. In some embodiments, the flow meters may also be capable of measuring temperature and/or density. The outputs of the flow meters may be inputted to controller 113 on a continuous or regular basis. Preferably, there is at least one flow meter upstream of each of the flow meter valves. In some embodiments, the flow meters and flow meter valves may be integral and provided together in a compact package. Although one controller is shown, in some embodiments there may be a plurality of controllers. As shown in FIG. 4, the AA powder is fed to loss-in-weight feeder 110 via line 102 to produce a metered AA powder feed 139. Controller 113 sends a signal 211 to rotating auger 123. The signals may be wireless signals. Using the model, the model for the feed forward feed rates for the HMD and water may be stored in controller 113. Loss-in-weight feeder 110 adjusts for the variability in AA powder, as described above, to provide a metered AA powder feed 139 having low variability from a target feed rate. For example, loss-in-weight feeder 110 may use a feedback loop from weight measuring subsystem 121 to adjust the speed of rotary auger 123.

Controller 113 sends a feed forward signal 213 to flow meter valve 214 to regulate the flow of water 103 into disperser 300. Similarly, controller 113 sends a feed forward signal 215 to flow meter valve 216 to regulate the flow of HMD 104 into disperser 300. These feed forward signals are set by the model to achieve the target pH, molar ratio of AA to HMD, and/or target salt concentration. HMD and water may be combined as an HMD aqueous solution and fed to disperser 300.

In another embodiment, controller 113 sends a feed forward signal 227 to flow meter valve 228 to regulate the feed rate of PBA solution 306 into continuous stirred tank reactor 140. When no storage tank 184 is used, flow meter valve 228 must be set to the production rate of vessel 302, which may limit inventories. These feed forward signals are set by the model to achieve the target pH and target salt concentration. Because the feed forward signals 213 and 215 are used for the HMD and water to disperser 300, it is not necessary to take any on-line or off-line measurements of PBA solution 306. To supply the sufficient amount of HMD and water to form the desired nylon salt solution, DCS controller 113 may send separate feed forward signals to HMD and water fed to continuous stirred tank reactor 140 based on the feed rate of PBA solution 306 to continuous stirred tank reactor 140. Feed forward signal 229 may be based on the target feed rate of PBA solution 306, and feed forward signal 229 may control flow meter valve 230 to supply balance HMD 104′ to continuous stirred tank reactor 140. In addition, there is a feed forward signal 217 to flow meter valve 218 to regulate the flow of trim HMD 107 into recirculation loop 141. The model may determine the relative amounts of HMD fed through the HMD 104 to disperser 300, main HMD 104′ and trim HMD 107. Controller 113 may also send a feed forward signal 231 may control flow meter valve 232 to supply trim water 103′ to continuous stirred tank reactor 140. Trim water 103′ may be supplied directly to continuous stirred tank reactor 140 or through a vent line. Feed forward signal 217 and feed forward signal 229 are adjusted to insure that there is a midrange output flow to flow meter valve 217 of trim HMD 107. In one embodiment, the model may establish a feed rate that is transmitted by feed forward signal 217 to flow meter valve 218 to ensure that a constant flow, i.e. midrange flow, is maintained from trim HMD 107.

### Secondary Process Controls

In addition to using feed forward controls based on modeling, as shown in FIG. 4, the process controls may include feedback signals as secondary process controls to achieve the target pH and target salt concentration. These feedback signals may be measurements taken from flow meters and on-line analyzer 154 that are used to adjust the HMD and water feeds, preferably trim HMD and water feeds. On-line analyzers 154 may be include pH probes, refractometers, and combinations thereof. The pH probes and refractometers may be in series or in parallel.

As shown in FIG. 4, the process generates feedback signals using an on-line analyzer 154, e.g., on-line pH meter 154 to measure the pH of the nylon salt solution in recirculation loop 141. To facilitate the on-line measurement of the pH of the nylon solution, the nylon salt solution is continuously withdrawn from the reactor and at least a portion of the nylon salt solution is directed to recirculation loop 141 and to sample line 153. Recirculation loop 141 may comprise a flow meter (not shown) and flow meter valve. In another embodiment, recirculation loop 141 may comprise a pressure controller (not shown) to control flow of the nylon salt solution. Preferably, the flow of nylon salt solution through recirculation loop 141 is constant. Sample line 153 comprises a means for pH measurement, e.g., a pH meter and/or a means for salt concentration measurement, e.g., a refractometer. In one embodiment, the pH of the at least a portion of the nylon salt solution is measured at reactor conditions, without any dilution or cooling. The at least a portion of the nylon salt solution is then returned to reactor 140, either directly or through vent condenser 131. When the at least a portion of the nylon salt solution is returned to the reactor through vent condenser 131, the nylon salt solution may replace water fed to the vent condenser. Sample line 153 may also comprise a cooler (not shown) to cool the nylon salt solution and a temperature sensor (not shown) to measure the temperature prior to pH measurement. In some embodiments, the nylon salt solution is cooled to a target temperature prior to pH measurement. This target temperature may be a target within the range between 5° C. and 10° C. cooler than the nylon salt solution as it exits reactor 140. The temperature may vary by less than ±1° C. from the target temperature, e.g., by less than ±0.5° C. There may be a temperature sensor (not shown) to monitor the temperature of the nylon salt solution upstream of the pH measurement.

On-line pH meter 154 then provides output 226 to controller 113. This output 226 transmits the pH value measured in on-line pH meter 154 to controller 113. On-line pH meter 154 is used to determine the variability of the pH of the nylon salt solution during a continuous process. In other words, on-line pH meter 154 may measure a pH that is different than the target pH, but controller 113 adjusts monomer feeds when the measured pH has variations. In preferred embodiments, the pH of the nylon salt solution varies by less than ±0.04, e.g., less than ±0.03 or less than ±0.015. Because of drift in on-line pH meter measurement values, the on-line pH meter is used to measure variability of pH instead of an absolute pH value. This is due at least in part to the feed forward controls which allow for a target pH to be set. By using the on-line pH meter to determine if the pH varies, changes in the production process may be detected. Using the secondary controls, a change in pH may cause a corresponding adjustment of at least one of the feed rates sent via signal lines 217 and 229 to flow meter valves 218 and 230 respectively. In one aspect, when PBA solution 306 is fed at a constant amount to reactor 140, it is preferred to adjust feed rates of HMD and water to reactor 140 instead of adjusting feed rates to the disperser 300 that produces the PBA solution. To provide a responsive pH adjustment, a signal is sent via line 217 to valve 218 to adjust the trim HMD 107. The amount of adjustment made to trim HMD 107 may be accounted for by a corresponding change to the HMD 104′ by flow meter valve 230. It is less preferred to adjust the HMD 104 to vessel 300 since this will impact the PBA solution. This adjustment is responsive and should be able to revert to the feed rates set by the feed forward controls once pH variation is not shown. These adjustments to trim HMD 107 may also affect the salt concentration of the nylon salt solution. Such salt concentration changes may be controlled by adjusting the water via signal 231 through flow meter valve 232.

Because the process described to form the nylon salt solution is continuous, the pH measurements in on-line pH meter 154 may be obtained in real time (e.g., continuously) or in near real time. In some embodiments, the pH measurement is taken every 60 minutes, e.g., every 45 minutes, every 30 minutes, every 15 minutes, or every 5 minutes. The pH meter may have accuracy to within ±0.05, e.g., ±0.02.

The process may also further comprise measuring the salt concentration in the nylon salt solution using a refractometer in addition to on-line pH meter 154, and adjusting the water feed rates. In one embodiment, the water feed rates may be adjusted by the water feed to recovery column 131. The salt concentration may also be adjusted by adding or removing water from the nylon salt solution downstream of the reactor.

Depending on the adjustment needed based on the feedback, the secondary controls may also be used by the model to independently adjust the main HMD and water to both the disperser and reactor. This is particularly advantageously when there is a pH trend that causes a long-term adjustment of the trim HMD 107.

In addition to the feedback from on-line pH meter 154, each flow meter, 214′, 215′, 218′, 228′, 230′, and/or 232′, may provide information, or mass flow rates, to controller 113 via signals 213′, 215′, 218′, 227′, 229′, and/or 231′, respectively. This information from the flow meter may be used to maintain overall production rates.

Prior art processes using pH probes to measure the pH of a nylon salt solution have been disclosed. See U.S. Pat. No. 4,233,234 and U.S. Pub. No. 2010/0168375. However, each of these prior art processes measure the pH of the nylon salt solution and then add additional diamine and/or acid to adjust the pH. The effect of the additional diamine and/or acid is not determined until the additional diamine and/or acid is blended into the reactor and withdrawn again for measurement. This method results in “chasing” the pH and creates an unresponsive process control that may overshoot or undershoot the target pH.

In the present invention, as shown in FIG. 4, trim HMD 107 is preferably fed upstream of the on-line pH meter. Thus, the HMD in trim HMD 107 is combined with the nylon salt solution in the reactor recirculation loop and the pH of the nylon salt solution is measured prior to being recirculated through reactor 140.

Secondary Process Controls with On-Line Laboratory Measurement

As stated above, the pH measurement from the secondary process control is not necessarily reflective of the target pH, but rather is used to account for pH variations. To improve the sensitivity of the pH measurement, the secondary process controls may also involve measuring the pH of the nylon salt solution under laboratory controls. Without being bound by theory, measuring the pH of the nylon salt solution under laboratory conditions improves the accuracy of the measurement due to increased sensitivity of pH measurements near the inflection point under conditions of reduced concentration and temperature. This may allow detection of small pH changes that might not be noticed under reaction conditions. For purposes of the present invention, laboratory conditions refer to measuring the nylon salt solution sample at a temperature between 15° C. and 40° C., e.g., between 20° C. and 35° C. or at 25° C., ±0.2° C. The nylon salt solution sample measured under laboratory conditions may have a salt concentration between 8 and 12%, e.g., 9.5%. This pH measurement under laboratory conditions is made on-line by diluting and cooling the nylon salt solution in sample line 153.

As shown in FIG. 4, to facilitate the on-line measurement of the pH of the nylon solution at laboratory conditions, the nylon salt solution is continuously withdrawn from the reactor and at least a portion of the nylon salt solution, e.g., less than 1%, is directed to recirculation loop 141 and to sample line 153. Sample line 153 comprises a means for pH measurement under laboratory conditions. Sample line 153 may also comprise a cooler (not shown) to cool the nylon salt solution. In other embodiments, this cooler may be omitted. The temperature and concentration of the nylon salt solution in sample line 153 may be adjusted by adding water via line 220. This water is a small part of the total water feed rate that is accounted by the model. The water is added in an amount and at a temperature sufficient to reach the desired temperature and strength of diluted nylon salt solution sample for pH measurement. Further cooling of the diluted sample may be included. The pH of the at least a portion of the nylon salt solution is taken at laboratory conditions and the at least a portion of the nylon salt solution is then returned to reactor 140 as described herein. On-line pH meter 154 then provides output 226 to controller 113.

As described above, on-line pH meter 154 is used to measure variability in pH of the nylon salt solution. In preferred embodiments, the pH of the nylon salt solution varies by less than ±0.04, e.g., less than ±0.03 or less than ±0.015. Similar to the pH measurements at reaction conditions, because of drift in on-line pH meter measurement values, the on-line pH meter under laboratory conditions is used to measure variability of pH instead of the target pH. This is due at least in part to the feed forward controls which allow for a target pH to be set. By using the on-line pH meter to determine if the pH varies, changes in the production process may be detected. Similar to the secondary process controls, the feed rates may be adjusted by sending a signal to lines 217 and 229 to flow meter valves 218 and 230. These adjustments may also affect the salt concentration of the nylon salt solution. Such salt concentration changes may be controlled by adjusting the water via signal 231 to flow meter valve 232.

Because the process described to form the nylon salt solution is continuous, the pH measurements in on-line pH meter 154 may be obtained in real time (e.g., continuously) or in near real time. In some embodiments, the pH measurement is taken every 60 minutes, e.g., every 45 minutes, every 30 minutes, every 15 minutes, or every 5 minutes. The pH measurement means should have accuracy to ±0.05, e.g., ±0.03 or ±0.01.

### Tertiary Process Controls

Although the use of feed forward controls and feedback signals as shown in FIG. 4 may assist in reducing the variability of the nylon salt solution specifications, further analysis, particularly off-line pH analysis conducted at laboratory conditions, may be used to detect nylon salt solution uniformity. These off-line process controls at laboratory conditions, referred to as tertiary process controls, may include pH and/or salt concentration measurements. In one embodiment, the pH of the nylon salt solution may be measured off-line at laboratory conditions to determine whether the target pH is being achieved. The off-line pH measurements may also detect any instrument problems or biasing that may be adjusted. In another embodiment, the pH of the nylon salt solution measured off-line at laboratory conditions may also be used to adjust signal lines 217 and 229 to flow meter valves 218 and 230. The off-line pH measurements under laboratory conditions may have a capacity measure pH to within ±0.01.

As described herein, laboratory conditions refer to measuring the nylon salt solution sample at a temperature between 15 and 40° C., e.g., between 20 and 35° C. or at 25° C., ±0.2° C., e.g., ±0.2° C. The nylon salt solution sample measured under laboratory conditions may have a concentration between 8 and 12%, e.g., 9.5%. To reach this temperature and concentration, the nylon salt solution sample removed from the recirculation loop may be diluted and cooled with water. A temperature bath may be used to cool the diluted nylon salt solution sample. The sample may be withdrawn on an as-needed basis, such as every 4 to 6 hours, daily or weekly. In the case of a system upset, the sample may be withdrawn more frequently, e.g., hourly. In general, the off-line analyzer may be used to account for instrument bias of the on-line analyzer. For example, if the target pH is 7.500, the on-line analyzer may report a pH of 7.400 while the off-line analyzer reports a pH of 7.500, indicating an on-line pH analyzer instrument bias. In one aspect, an exponential weighted moving average may be used to automatically bias the on-line analyzers each time an off-line measurement is made. In some aspects, the output of the off-line analyzer is used to correct any bias or drift in the on-line analyzer. In other aspects, the on-line analyzer is not corrected but the drift or bias is monitored by the off-line analyzer. In this aspect, the on-line analyzer is relied upon to determine variation in pH, e.g., outside of a preset acceptable variability.

In another embodiment, an off-line analyzer may be used to measure the target salt concentration of the nylon salt solution. The off-line salt concentration measurements may also detect any instrument problems or biasing that may be adjusted. Each refractometer may be independently biased when multiple refractometers are used.

### Nylon Polymerization

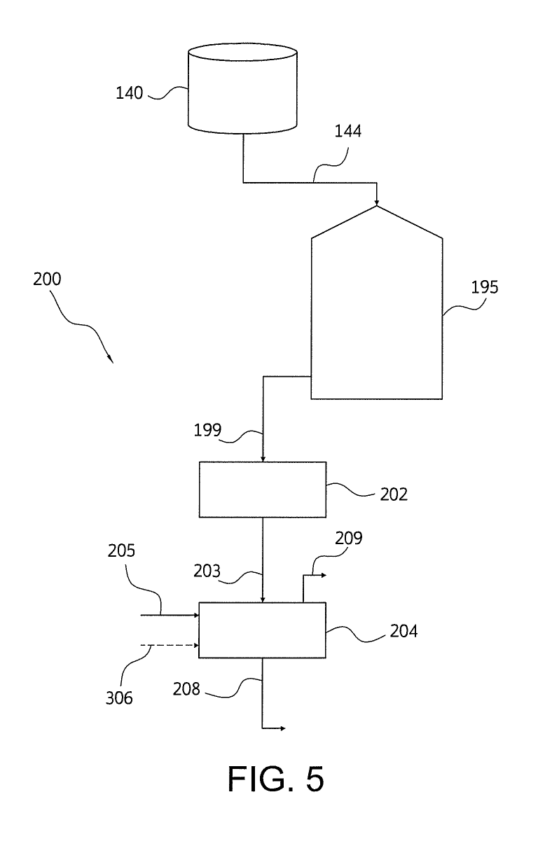

The nylon salt solution described herein may be directed to polymerization process 200 to form a polyamide, in particular nylon 6,6. The nylon salt solution may be sent directly from the continuous stirred tank reactor 140 to a polymerization process 200 or may first be stored in a storage tank 195 and then sent to polymerization process 200, as is shown in FIG. 5.

The nylon salt solution of the present invention has a uniform pH that improves the performance of the polyamide polymerization process. The uniform pH of the nylon salt solution provides a reliable starting material to produce various polyamide products. This greatly improves the reliability of the polymer product. In general, the polymerization process comprises evaporating water from the nylon salt solution to concentrate the nylon salt solution and polymerizing the concentrated nylon salt via condensation to form the polyamide product. One or more evaporators 202 may be used. The evaporating of water may be done in a vacuum or under pressure to remove at least 75% of the water in the nylon salt solution, and more preferably at least 95% of the water in the nylon salt solution. The concentrated nylon salt 203 may comprise between 0 and 20 wt. % water. The condensation may be carried out in a batch or continuous process. Depending on the desired end polymer product, additional AA and/or HMD may be added to the polymerization reactor 204. In some embodiments, additives may be combined with the polyamide product.

For purposes of the present invention, suitable polyamide products may have at least 85% of the carbon chains are aliphatic between the amide groups.

The nylon salt solution may be maintained at a temperature above its melt pointing when being transferred from the storage tank 195 to evaporators 202. This prevents fouling of the lines. In some embodiments, steam captured from evaporators 202 may be used to maintain the temperature. In other embodiments, cooling water that is heated may also be used.

The polymerization may be in a single stage reactor or multi-stage condensation reactor 204. Additional monomers, either AA or HMD, but preferably HMD, may be added via line 205 to produce different nylon products 208. In one embodiment, a portion of the PBA solution 308 may be introduced to reactor 204 to produce different nylon products 208. Reactor 204 may comprise an agitator for mixing the nylon salt. Reactor 204 may also be jacketed using a heat transfer medium to regulate the temperature. The condensation reaction in reactor 204 may be carried out in an inert atmosphere and nitrogen may be added to reactor 204. The temperature of the polymerization may vary depending on the starting dicarboxylic acid and diamine, but is generally greater than the melt temperature of the nylon salt, and more preferably at least 10° C. above the melt temperature. For example, a nylon salt comprising hexamethylene diammonium adipate salt has a melt temperature within the range between 165° C. and 190° C. Thus, the condensation reaction may be conducted at a reactor temperature between 165° C. and 350° C., e.g., between 190° C. and 300° C. The condensation reaction may be carried out at atmospheric pressure or under a pressurized atmosphere. The nylon product 208 is removed from the reactor as a free-flowing solid product.

Water generated during the condensation reaction may be removed as a vapor stream through reactor vent line 209. The vapor stream may be condensed and vapor monomers, such as diamine, escaping with the water may be returned to the reactor.

Subsequent processing may be performed, e.g., extruding, spinning, drawing, or draw-texturing, to produce the polyamide product. The polyamide product may be selected from the group consisting of nylon 4,6; nylon 6,6; nylon 6,9; nylon 6,10; nylon 6,12; nylon 11; and nylon 12. In addition, the polyamide product may a copolymer, such as nylon 6/6,6.

The following non-limiting examples describe the process of this invention.

### EXAMPLES

### Example 1

AA powder is transferred from an unloading system by either bulk bag unloading, lined bulk bag unloading, lined box container unloading, or hopper railcar unloading stations by means of either mechanical (i.e. screw, drag chain) or pneumatic (i.e. pressure air, vacuum air, or closed loop nitrogen) conveyance system(s) to supply vessel.

The supply vessel transfers AA powder on demand to a loss-in-weight (L-I-W) feeder, and is regulated by a PLC based on selected L-I-W hopper low and high levels. The supply vessel meters AA powder by screw conveyor or rotary feeder at a sufficient loading rate to allow filling of the L-I-W feeder hopper at a maximum interval equal to one-half, and preferably less than one half, the minimum L-I-W discharge time from high to low level of the L-I-W bin, in order to receive feedback of L-I-W feeder feed rate at least 67% of the time.

The L-I-W feeder system PLC regulates the L-I-W feeder screw speed to maintain feed rate, as measured from the L-I-W feeder hopper load cells, at a feed rate target received from the Distributed Control System (DCS).

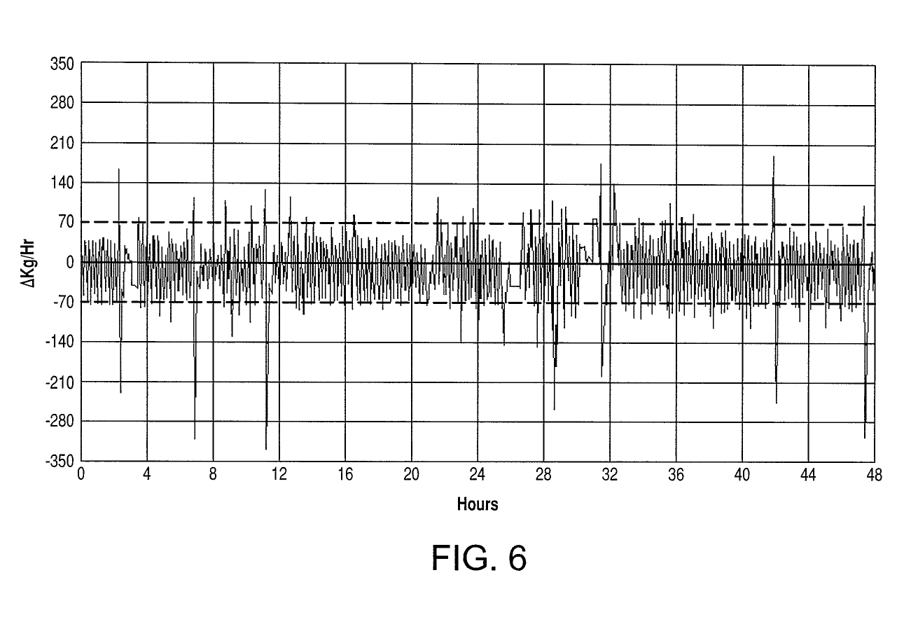

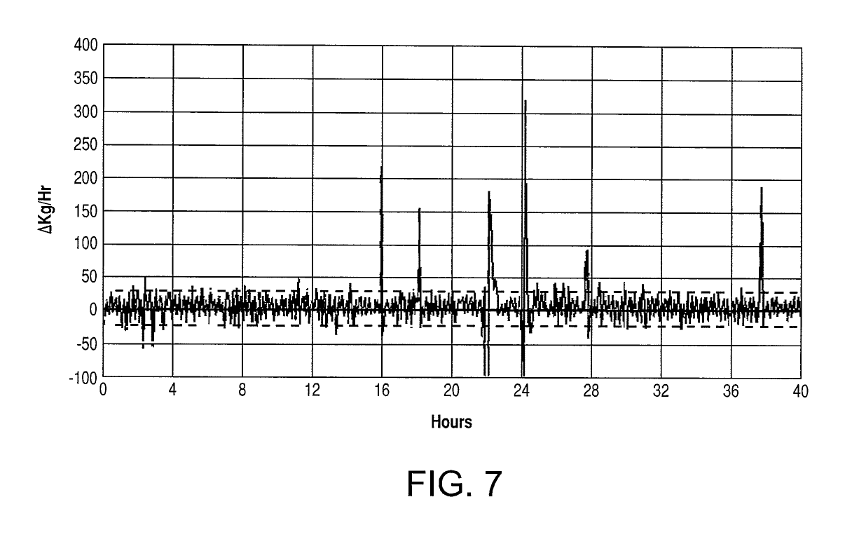

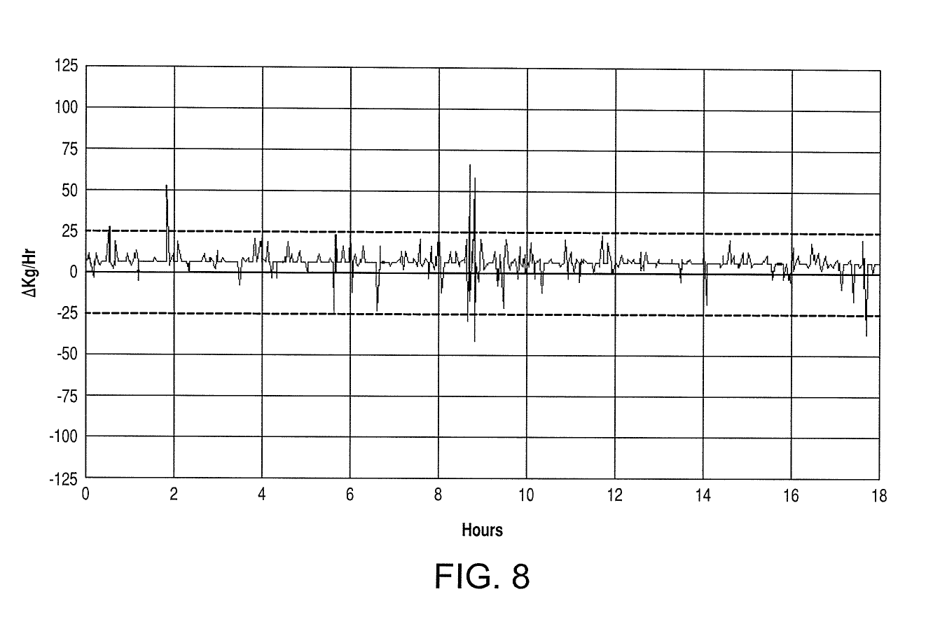

As shown in FIG. 6 the feed rate variability of adipic acid through a loss-in-weight feeder has a feed rate variability of less than ±5% over a 48 hour period of continuous feeding. As shown in FIG. 7, the feed rate variability may be less than ±3% over a 40 hour period. As shown in FIG. 8, the feed rate variability may be less than ±1% over an 18 hour period. Using a loss-in-weight feeder for adipic acid results in an improved feed rate variability performance by eliminating the disturbances to the adipic acid feed rate causes by using a volumetric feeder.

### Example 2

A partially balanced adipic (PBA) solution is prepared as follows.

This AA powder feed is supplied from a L-I-W feeder to an in-line disperser that continuously blends the reactant mixture of AA powder with a dilute HMD solution to produce a PBA solution having 42.6% AA, 14% HMD, and 43.4% de-ionized water. The partially balanced acid solution has a 56.7 wt. % solids concentrations and contains 25.1 wt. % free AA and 31.6 wt. % salt concentration.

The DCS set point for the L-I-W AA feed rate is determined by a DCS model based on PBA solution feed rate to the continuous stirred tank reactor (CSTR) and target inventory level for partially balanced acid solution storage.

The dilute HMD solution is prepared as follows. HMD solution (98%) is supplied to the in-line disperser from a pressure controlled HMD storage recirculating header. Using coriolis mass flow meter measurement with input to the DCS, the DCS regulates the HMD feed stream flow rate to the in-line disperser to accurately control the ratio of AA and HMD in the disperser product stream. For a 63% salt concentration target, the HMD charge is 41.2% of the required HMD charge to the process is added to the in-line disperser. HMD solution is fed at a temperature that is less than 45° C.

De-ionized water is supplied to the in-line disperser from a pressure controlled de-ionized water supply header. Using coriolis mass flow meter measurement with input to the DCS, the DCS regulates the de-ionized water feed stream flow rate to the in-line disperser to accurately control the aqueous concentration of AA and HMD in the disperser product stream. For a 63% salt concentration target, the partially balanced acid solution feed is a minimum of 56.75% solids (43.25% water) to allow for a minimum de-ionized water injection for the reactor's vent condenser and for concentration trim adjustment. Water is fed at a temperature near ambient temperatures of between 20° C. and 25° C.

In-line disperser product stream is mixed with aqueous partially balanced acid solution storage recycle upstream of the recirculation loop heat exchanger to elevate the partially balanced acid solution product stream temperature to a minimum of at least 50° C., preferably between 55° C. and 60° C., to maintain the partially balanced acid solution product stream as a homogeneous solution free of suspended crystals. The confluence of these two streams incorporates a liquid jet eductor (hereinafter “eductor”), with the recycle stream serving as the motive flow and the disperser discharge as the educted flow, in order to accommodate the required near-atmospheric discharge pressure of the in-line disperser and to facilitate blending with product in storage to maximize homogeneity. Alternately, a booster pump may be used in place of the eductor. The partially balanced acid solution storage recycle stream recirculation rate is controlled in order to provide sufficient motive flow rate to the re-circulating line and storage tank's mixing eductors. The tank mixing eductor is located between 0.2 and 1.5 meters from the tank bottom, e.g., preferably between 0.5 and 1 meter, in order to insure complete mixing of the in-line disperser product with the tank contents. Storage tank temperature is regulated between 50° C. to 60° C., preferably 55° C. to 60° C., by adjustment of the steam flow rate to the recirculation line heat exchanger.

### Example 3

A partially balanced adipic (PBA) solution is prepared as follows.

This AA powder feed is supplied from a L-I-W feeder to a vessel, in this case a continuous stirred tank reactor, comprising a disperser head. Disperser head continuously blends the reactant mixture of AA powder with a dilute HMD solution to produce a PBA solution having 43.3% AA, 14.2% HMD, and 42.5% de-ionized water. The vessel also has an external, in-line mixer for additional milling of the reactant mixture, with recirculation back to the disperser CSTR. The partially balanced adipic solution has a 57.5 wt. % solids concentrations and contains 25.4 wt. % free AA.

The DCS set point for the L-I-W AA powder feed rate is determined by a DCS model based on PBA solution feed rate to the CSTR and target inventory level for PBA storage.

The dilute HMD solution is prepared as follows. HMD solution (98%) is supplied to the PBA vessel from a pressure controlled HMD storage recirculating header. Using coriolis mass flow meter measurement with input to the DCS, the DCS regulates the HMD feed stream flow rate to the PBA vessel to accurately control the ratio of AA and HMD in the reactant mixture. For a 63% salt concentration target, the HMD charge to vessel is 41.2% of the required HMD charge to the process is added to the in-line disperser. HMD solution is fed at a temperature that is greater than 45° C.

De-ionized water mixes with the HMD solution to form the dilute HMD solution to the vessel. Water is supplied from a pressure controlled de-ionized water supply header. Using coriolis mass flow meter measurement with input to the DCS, the DCS regulates the de-ionized water feed stream flow rate to the vessel to accurately control the aqueous concentration of AA and HMD in the reactant mixture. For a 63% salt concentration target, the PBA solution feed is a minimum of 56.75% solids (43.25% water) to allow for a minimum de-ionized water injection for the reactor's vent condenser and for concentration trim adjustment. Water is fed at a temperature near ambient temperatures of between 20° C. and 30° C.

The PBA vessel also has an external in-line mixer for mixing the reactant mixture and recirculating the dispersion back to the vessel as well as for feeding the dispersion forward to the storage tank.

The in-line mixer is assisted by pump or supplied as the inducted flow to an in-line eductor in the recirculation loop of the storage tank, when limited in head capacity below the pressure required to discharge the dispersion directly to a subsequent tank. The storage tank's recirculation liquid is used as the motive flow for the in-line eductor.

A storage vessel recirculation loop heat exchanger is used in-line to elevate the PBA solution temperature to a minimum of 50° C., preferably 55° C. and 60° C., to maintain the PBA as a homogeneous solution, free of suspended crystals. The confluence of the dispersion from the vessel and storage tank recycle, either as a simple pipe tee with pressurized pump feed or with the in-line eductor with in-line disperser feed, is installed upstream of the recirculation loop heat exchanger to insure the blended stream has achieved minimum desired temperature prior to entering the storage vessel.

The PBA storage recycle stream recirculation rate is controlled in order to provide sufficient motive flow rate to the re-circulating line eductor, and storage tank's mixing eductors. The tank mixing eductor will be located between 0.2 and 1.5 meters from the tank bottom, e.g., preferably between 0.5 and 1 meter, to insure complete mixing of the dispersion with the tank contents. Storage tank temperature will be regulated between 50° C. and 60° C., preferably between 55° C. and 60° C., by adjustment of the steam flow rate to the recirculation line heat exchanger.

### Example 4

A nylon salt solution is prepared to achieve a target salt concentration of 63% concentration and a target pH of 7.500. The PBA solution prepared in Example 2 is used as the source of adipic acid for the nylon salt solution.

The DCS provides a target feed rate of PBA solution to the salt CSTR using a DCS model that is based on polymerizer production rate and target salt inventory level and adjusts the target at configurable intervals. The PBA solution feed rate is measured by means of a coriolis mass flow meter and controlled to a target in DCS.

The DCS uses a feed-forward ratio control loop to control the feed rate of balance HMD based on the target PBA feed rate. The set point of the DCS balance HMD ratio flow controller is adjusted to maintain the output of the trim HMD valve to midrange to insure the valve is in control range continuously. For 63% salt target, the balance HMD charge is normally 48.8% to 56.8% of the HMD charge to the process, and about 90 to 98% of the HMD charge when combined with the PBA solution feed HMD component.

The pH is continuously measured by redundant pH meters in a filtered, temperature & flow controlled sample recirculation loop supplied by the reactor's recirculation pump. Using the DCS selected pH input of the continuously compared pair of in-line pH measurements, the DCS regulates the feed rate of trim HMD in order to maintain pH to a target set point in DCS. For 63% salt target, the trim HMD charge is about 2 to 10% of the total HMD charged to the process.

The setpoint of the pH controller is adjusted based on a statistically based algorithm using pH analysis of samples that are taken at discrete intervals downstream of the reactor and which are conditioned to 9.5% concentration and 25° C. to achieve maximum sensitivity of acid/amine balance as a function of pH, or by continuous input of pH from an on-line analyzer that continuously dilutes/conditions the reactor's product, or product from a subsequent storage vessel if preferred, to 9.5% concentration and 25° C.

The trim HMD is injected into the main reactor recirculation loop pump suction in order to achieve fastest response time to the pH meters and to insure the reactor product is adjusted to target in the shortest time. The pump is used to blend the HMD and reactor salt product in order to insure the pH and concentration meters have a homogeneous solution for their respective measurements.

The reactor's concentration is continuously measured by redundant refractometers in the same filtered, temperature & flow controlled sample recirculation loop supplied by the reactor's recirculation pump. Using the DCS selected concentration input of the continuously compared pair of in-line concentration measurements, the DCS regulates the feed rate of trim de-ionized water in order to maintain concentration to a target set point in DCS. For 63% salt target, the trim water charge is 1 to 5%, preferably about 3%, of the total water charged to the process.

The reactor product is continuously fed, by means of level control of the CSTR, to storage tank where it is further blended for supply to the polymer plant. This transfer includes at least one bank of parallel arranged, cartridge type filter housings, designed for a maximum of 34.5 kPa (5 psig) initial clean pressure drop at maximum instantaneous salt solution transfer rate to storage. Cartridge removal efficiency is a minimum of 10 μm absolute rating with use of synthetic fiber depth or pleated membrane cartridges, or a minimum of 1 μm nominal rating when wound cotton fiber cartridges are used. Filter selection is based on alternatives with rating for a minimum of 110° C. operating temperature.

The aqueous salt is continuously recirculated through salt storage tank(s), with preference for use of tank mixing eductors, located between 0.5 and 1 meter from the tank bottom, for more rapid turnover of tank contents to maximize blending efficiency.

For 63% salt concentration, salt storage tank temperature is regulated between 100° C. to 105° C. by adjustment of the steam flow rate to the recirculation line heat exchanger. The salt in the storage tank has a uniform pH of 7.500 that is less than ±0.0135 from the target pH.

The nylon salt solution may also be prepared using the PBA solution of Example 3 to obtain the nylon salt solution having a target pH and a target salt concentration.

### Comparative Example A

A mixture was prepared from Example 1 of U.S. Pat. No. 6,995,233. A concentrated aqueous HMD solution with a concentration by mass of water equal to 10%, and of AA powder are fed continuously into a first stirred reactor to obtain a mixture having a weight ratio of 81% of AA monomer and 19% diamine monomer. This mixture may contain a small amount of water, for example about 7% by weight relative to the AA/HMD mixture. The mixture is maintained at a temperature of about 126° C. to prevent crystallization.

### Comparative Example B

The model and process are followed as in Examples 1-2, except that no HMD is fed to the in-line disperser. The partially balanced acid solution from the storage tank comprises 49.7 wt. % adipic acid and 50.3 wt. % water and must be maintained at a temperature above 85° C. to prevent solidification.

### Comparative Example C

The model and process are followed as in Examples 1-2, except that no water is fed to in-line disperser. Feeding only AA and HMD in-line disperser is not possible because the in-line disperser is not capable of dissolving AA without water. The product would have a high viscosity and would be manageable only at much higher temperatures.

### Comparative Example D

The model and process are followed as in Examples 1-2, except that instead of using L-I-W feeder, a volumetric feeder is used to feed AA powder to the in-line disperser. The pH of the nylon salt solution varies by greater than 0.1 pH units from the target pH. The poor control in the pH may result in a significantly higher freeze point that would require a higher processing temperature to prevent risk of crystallization.

### Comparative Example E

The model and process are followed as in Examples 1-2 and 4, except that no trim water is fed to the reactor. Salt concentration in the nylon salt solution increases from 63% to 63.707% which requires a higher storage temperature, e.g., 3.5° C. to 4° C., prior to polymerization. The increased temperature for storage would be closer to the boiling point temperature at atmospheric pressure for the nylon salt solution. To compensate for the increased salt concentration, the concentration of the partially balanced acid solution is reduced and because there is no trim water to adjust, and it would be more difficult to achieve a uniform concentration.

### Comparative Example F

The model and process are followed as in Examples 1-2, except that at the convergence of dispersion discharge and recirculation loop there is no eductor or booster pump. A loss in motive flow reduces the eductor blending efficiency and also loses the vacuum potential that enables the in-line disperser to discharge without backpressure. A further more serious issue is that the dispersion discharge will not have a sufficient head pressure to match the salt storage tank recirculation header pressure. Due to the pressure drop the dispersion discharge has an insufficient pressure to enter storage tank.

While the invention has been described in detail, modifications within the spirit and scope of the invention will be readily apparent to those skilled in the art. All publications and references discussed above are incorporated herein by reference. In addition, it should be understood that aspects of the invention and portions of various embodiments and various features recited may be combined or interchanged either in whole or in part. In the foregoing descriptions of the various embodiments, those embodiments which refer to another embodiment may be appropriately combined with other embodiments as will be appreciated by one skilled in the art. Furthermore, those skilled in the art will appreciate that the foregoing description is by way of example only, and is not intended to limit the invention.

1. A process for controlling the continuous preparation of a nylon salt solution comprising:
a) generating a model for setting a target feed rate of dicarboxylic acid powder to produce the nylon salt solution having a target pH;
b) controlling feed rate variability of the dicarboxylic acid powder by metering the dicarboxylic acid powder, based on weight, from a loss-in-weight feeder to a feeding conduit that transfers the dicarboxylic acid powder at the target feed rate into a disperser;
c) separately introducing diamine at a first feed rate and water at a second feed rate to the disperser, wherein the first and/or the second feed rates are based on the model, to produce a partially balanced salt solution;
d) introducing the partially balanced salt solution at a third feed rate, diamine at a fourth feed rate and water at a fifth feed rate to a single continuous stirred tank reactor, wherein the third, fourth and/or fifth feed rates are based on the model; and
e) continuously withdrawing the nylon salt solution from the single continuous stirred tank reactor directly into a storage tank, wherein the withdrawn nylon salt solution has a pH less than ±0.04 of the target pH.

2. The process of claim 1, wherein the disperser is an in-line disperser.

3. The process of claim 1, wherein the disperser is a vessel with a disperser head.

4. The process of claim 1, wherein the partially balanced acid solution comprises between 32 wt. % and 46 wt. % dicarboxylic acid, between 11 wt. % and 15 wt. % diamine, and between 39 wt. % and 57 wt % water.

5. The process of claim 1, wherein the target feed rate of dicarboxylic acid powder is based on the production rate for the nylon salt solution.

6. The process of claim 1, wherein the stoichiometric amount of the dicarboxylic acid powder need to produce the nylon salt solution is fed to the disperser.

7. The process of claim 1, further comprising maintaining the temperature of the partially balanced acid solution at a temperature between 50° C. and 60° C., preferably between 50° C. and 55° C.

8. The process of claim 1, further continuously introducing a trim diamine feed at a sixth feed rate to a recirculation loop of the single continuous stirred tank reactor, wherein the sixth feed rate is based on the model.

9. The process of claim 8, further comprising:
f) detecting a change in pH of the nylon salt solution using an on-line pH measurement of the nylon salt solution downstream of the trim diamine introduction; and
g) adjusting the sixth feed rate in response to the change in pH to produce the nylon salt solution having a pH that varies by less than ±0.04 from the target pH.

10. The process of claim 8, further comprising:
f) obtaining a sample portion of the nylon salt solution downstream of the trim diamine introduction;
g) diluting and cooling the sample portion to form a diluted nylon salt solution having a concentration between 5% and 15% and a temperature between 15° C. and 40° C.;
h) detecting a change in pH of the diluted nylon salt solution using an on-line pH measurement of the nylon salt solution downstream of the trim diamine introduction; and
i) adjusting the sixth feed rate in response to the change in pH from the diluted nylon salt solution.

11. The process of claim 8, further comprising:
f) removing a sample from the nylon salt solution downstream of the trim diamine introduction for an off-line pH measurement of the nylon salt solution in an aqueous solution at a temperature between 15 and 40° C.;
g) determining a bias of an on-line pH measurement with the off-line pH measurement;
h) detecting a change in pH of the nylon salt solution using the biased on-line pH measurement of the nylon salt solution downstream of the trim diamine introduction; and
i) adjusting the sixth feed rate in response to the change in pH to produce the nylon salt solution having a pH that varies by less than ±0.04 from the target pH.

12. The process of claim 1, further producing the nylon salt solution having a target salt concentration that is selected from within the range between 50 wt. % and 65 wt. % comprising the steps of:
f) measuring the salt concentration in the nylon salt solution in the recirculation loop with one or more refractometers downstream of the trim diamine introduction; and
g) adjusting the fifth feed rate to control salt concentration of the nylon salt solution based on a target salt concentration, wherein the salt concentration of the nylon salt solution varies by less than ±0.5% from the target salt concentration.

13. The process of claim 1, wherein the target pH is selected from within the range between 7.200 and 7.900.

14. The process of claim 1, wherein the pH of the partially balanced acid solution is not measured prior to introducing the partially balanced acid solution into the single continuous stirred tank reactor.

15. The process of claim 1, wherein the dicarboxylic acid is adipic acid and the diamine is hexamethylene diamine and wherein the nylon salt solution comprises hexamethylene diammonium adipate salt.
